(19) United States
(12) Patent Application Publication
(10) Pub. No.: US 2006/0094716 A1
(43) Pub. Date: May 4, 2006

(54) 1-Pyridin-4-yl-urea derivatives

(75) Inventors: Hamed Aissaoui, Pulversheim (FR); Christoph Binkert, Basel (CH); Boris Mathys, Egerkingen (CH); Claus Mueller, Hegenheim (FR); Oliver Nayler, Arlesheim (CH); Michael Scherz, Ettingen (CH); Thomas Weller, Biningen (CH); Jorg Welker, Lorrach (DE); Martine Clozel, Binningen (CH)

Correspondence Address: DICKSTEIN SHAPIRO MORIN & OSHINSKY LLP, 1177 AVENUE OF THE AMERICAS (6TH AVENUE), 41 ST FL., NEW YORK, NY, 10036-2714, US

(73) Assignee: Actelion Pharmaceuticals LTD, Allschwil (CH)

(21) Appl. No.: 10/528,043

(22) PCT Filed: Sep. 12, 2003
(86) PCT No.: PCT/EP03/10154

§ 371 Date: Mar. 17, 2005

(30) Foreign Application Priority Data
Sep. 17, 2002 (WO) PCT/EP02/10417

(51) Int. Cl. A61K 31/5377; A61K 31/4745; A61K 31/4709; A61K 31/4545; C07D 413/02; C07D 401/02; C07D 471/02

(52) U.S. Cl. 514/232.5; 514/313; 514/352; 514/300; 514/235.2; 514/318; 544/124; 544/128; 546/122; 546/159; 546/276.4; 546/306

(57) ABSTRACT

The invention relates to novel 1-pyridin-4-yl urea derivatives and related compounds and their use as active ingredients in the preparation of pharmaceutical compositions. The invention also concerns related aspects including processes for the preparation of the compounds, pharmaceutical compositions containing one or more of those compounds and especially their use as neurohormonal antagonists.

### FIELD OF THE INVENTION

The present invention relates to novel 1-pyridin-4-yl urea derivatives of the general formula 1 and their use as active ingredients in the preparation of pharmaceutical compositions. The invention also concerns related aspects including processes for the preparation of the compounds, pharmaceutical compositions containing one or more compounds of the general formula 1 and especially their use as neurohormonal antagonists.

### BACKGROUND OF THE INVENTION

Urotensin II is a cyclic 11-amino acid peptide neurohormone considered to be the most potent vasoconstrictor known, up to 28-fold more potent than endothelin-1. The effects of urotensin II are mediated through activation of a G-protein coupled receptor, the UT receptor, also known as GPR14 or SENR (Ames R S, et al, “Human urotensin-II is a potent vasoconstrictor and agonist for the orphan receptor GPR14” Nature (1999) 401, 282-6. Mori M, Sugo T, Abe M, Shimomura Y, Kurihara M, Kitada C, Kikuchi K, Shintani Y, Kurokawa T, Onda H, Nishimura O, Fujino M. “Urotensin II is the endogenous ligand of a G-protein-coupled orphan receptor, SENR (GPR14)” Biochem. Biophys. Res. Commun. (1999) 265,123-9. Liu Q, Pong S S, Zeng Z, et al, “Identification of urotensin II as the endogenous ligand for the orphan G-protein-coupled receptor GPR14” Biochem. Biophys. Res. Commun. (1999) 266, 174-178.) Urotensin II and its receptor are conserved across evolutionarily distant species, suggesting an important physiological role for the system (Bern H A, Pearson D, Larson B A, Nishioka R S. “Neurohormones from fish tails: the caudal neurosecretory system. I. Urophysiology and the caudal neurosecretory system of fishes” Recent Prog. Horm. Res. (1985) 41, 533-552). In euryhaline fish, urotensin II has an osmoregulatory role, and in mammals urotensin II exerts potent and complex hemodynamic actions. The response to urotensin II is dependent on the anatomical source and species of the tissue being studied. (Douglas S A, Sulpizio A C, Piercy V, Sarau H M, Ames R S, Aiyar N V, Ohlstein E H, Willette R N. “Differential vasoconstrictor activity of human urotensin-II in vascular tissue isolated from the rat, mouse, dog, pig, marmoset and cynomolgus monkey” Br. J. Pharmacol. (2000) 131, 1262-1274. Douglas, S A, Ashton D J, Sauermelch C F, Coatney R W, Ohlstein D H, Ruffolo M R, Ohlstein E H, Aiyar N V, Willette R “Human urotensin-II is a potent vasoactive peptide: pharmacological characterization in the rat, mouse, dog and primate” J. Cardiovasc. Pharmacol. (2000) 36, Suppl 1:S163-6).

Like other neurohormones, urotensin II has growth stimulating and profibrotic actions in addition to its vasoactive properties. Urotensin II increases smooth muscle cell proliferation, and stimulates collagen synthesis (Tzandis A, et al, “Urotensin II stimulates collagen synthesis by cardiac fibroblasts and hypertrophic signaling in cardiomyocytes via G(alpha)q- and Ras-dependent pathways” J. Am. Coll. Cardiol. (2001) 37, 164A. Zou Y, Nagai R, and Yamazaki T, “Urotensin II induces hypertrophic responses in cultured cardiomyocytes from neonatal rats” FEBS Lett (2001) 508, 57-60). Urotensin II regulates hormone release (Silvestre R A, et al, “Inhibition of insulin release by urotensin II-a study on the perfused rat pancreas” Horm Metab Res (2001) 33, 379-81). Urotensin II has direct actions on atrial and ventricular myocytes (Russell F D, Molenaar P, and O'Brien D M “Cardiostimulant effects of urotensin-II in human heart in vitro” Br. J. Pharmacol. (2001) 132, 5-9). Urotensin II is produced by cancer cell lines and its receptor is also expressed in these cells. (Takahashi K, et al, “Expression of urotensin II and urotensin II receptor mRNAs in various human tumor cell lines and secretion of urotensin II-like immunoreactivity by SW-13 adrenocortical carcinoma cells” Peptides (2001) 22, 1175-9; Takahashi K, et al, “Expression of urotensin II and its receptor in adrenal tumors and stimulation of proliferation of cultured tumor cells by urotensin II” Peptides (2003) 24, 301-306; Shenouda S, et al, “Localization of urotensin-II immunoreactivity in normal human kidneys and renal carcinoma” J Histochem Cytochem (2002) 50, 885-889). Urotensin II and its receptor are found in spinal cord and brain tissue, and intracerebroventricular infusion of urotensin II into mice induces behavioral changes (Gartlon J, et al, “Central effects of urotensin-II following ICV administration in rats” Psychopharmacology (Berlin) (2001) 155,426-33).

Dysregulation of urotensin II is associated with human disease. Elevated circulating levels of urotensin II are detected in hypertensive patients, in heart failure patients, in diabetic patients, and in patients awaiting kidney transplantation (Totsune K, et al, “Role of urotensin II in patients on dialysis” Lancet (2001) 358, 810-1; Totsune K, et al, “Increased plasma urotensin II levels in patients with diabetes mellitus” Clin Sci (2003) 104, 1-5; Heller J, et al, “Increased urotensin II plasma levels in patients with cirrhosis and portal hypertension” J Hepatol (2002) 37, 767-772).

Substances with the ability to block the actions of urotensin II are expected to prove useful in the treatment of various diseases. WO-2001/45694, WO-2002/78641, WO-2002/78707, WO-2002/79155, WO-2002/79188, WO-2002/89740, WO-2002/89785, WO-2002/89792, WO-2002/89793, WO-2002/90337, WO-2002/90348 and WO-2002/90353 disclose certain sulfonamides as urotensin II receptor antagonists, and their use to treat diseases associated with a urotensin II imbalance. WO-2001/45700 and WO-2001/45711 disclose certain pyrrolidines or piperidines as urotensin II receptor antagonists and their use to treat diseases associated with a urotensin II imbalance. These derivatives are different from the compounds of the present invention as they do not comprise urea derivatives bearing a 4-pyridinyl-like moiety. WO-2002/047456 and WO-2002/47687 disclose certain 2-amino-quinolones as urotensin II receptor antagonists and their use to treat diseases associated with a urotensin II imbalance. WO-2002/058702 discloses certain 2-amino-quinolines as urotensin II receptor antagonists and their use to treat diseases associated with a urotensin II imbalance. These derivatives are different from the compounds of the present invention as they do not bear a substituted urea function in the 4-position of the quinoline ring. WO-2001/66143 discloses certain 2,3-dihydro-1H-pyrrolo[2,3-b]quinolin-4-ylamine derivatives useful as urotensin II receptor antagonists, WO-2002/00606 discloses certain biphenyl compounds useful as urotensin II receptor antagonists, and WO-2002/02530 also discloses certain compounds useful as urotensin II receptor antagonists.

EP 428434 discloses certain alkylureidopyridines as neurokinin and substance P antagonists. WO-99/21835 discloses certain ureidoquinolines as H+-ATPase and bone resorption inhibitors. WO-01/009088 discloses certain substituted heteroarylureas as inhibitors of the CCR-3 receptor. All of these ureidopyridine derivatives differ in their composition from compounds of the present invention. The present invention comprises 1-pyridin-4-yl urea derivatives which are novel compositions of matter and which are useful as urotensin II receptor antagonists.

### DESCRIPTION OF THE INVENTION

The present invention relates to compounds of the general formula 1, 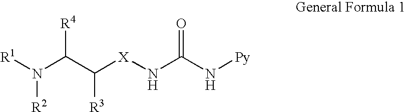
wherein:

Py represents quinolin-4-yl which is unsubstituted or mono- or disubstituted independently with lower alkyl or aryl-lower alkyl in the positions 2, 6 or 8; [1,8]naphthyridin-4-yl which is unsubstituted or monosubstituted in position 7 with lower alkyl; pyridin-4-yl which is unsubstituted or disubstituted in positions 2 and 6, whereby the substituent in position 2 is R⁵R⁶N—, lower alkyl, aryl-lower alkyl, or (E)-2-aryl-ethen-1-yl and the substituent in position 6 is hydrogen or lower alkyl;

X is absent or represents a methylene group;

R¹ represents hydrogen; lower alkyl; aryl; aryl-lower alkyl; lower alkyl disubstituted with aryl; or lower alkyl disubstituted with aryl and additionally substituted at a carbon atom bearing an aryl group with OH, CN, or CONR⁷R⁸;

R² forms together with R³ a five-, six-, or seven-membered ring containing the nitrogen atom to which R² is attached as a ring atom and in which case R⁴ represents hydrogen; or

R² forms together with R⁴ a five-, six-, or seven-membered ring containing the nitrogen atom to which R² is attached as a ring atom and in which case R³ represents hydrogen;

the rings formed between R² and R³ or between R² and R⁴ are unsubstituted or monosubstituted with lower alkyl, aryl, aryl-lower alkyl, hydroxy, or aryloxy;

R⁵ and R⁶ independently represent hydrogen; lower alkyl; aryl; aryl-lower alkyl; or form together with the nitrogen atom to which they are attached a pyrrolidine, piperidine, or morpholine ring;

R⁷ and R⁸ independently represent hydrogen; lower alkyl; aryl; aryl-lower alkyl; or form together with the nitrogen atom to which they are attached a pyrrolidine, piperidine, or morpholine ring;

and optically pure enantiomers or diastereomers, mixtures of enantiomers or diastereomers, diastereomeric racemates, and mixtures of diastereomeric racemates; as well as their pharmaceutically acceptable salts, solvent complexes, and morphological forms.

In the definitions of the general formula 1 the expression ‘lower alkyl’ means straight or branched chain groups with one to seven carbon atoms, preferably 1 to 4 carbon atoms. Lower alkyl also encompasses cyclic alkyl groups with three to six carbon atoms. Preferred examples of lower alkyl groups are methyl, ethyl, n-propyl, isopropyl, n-butyl, iso-butyl, sec-butyl, tert-butyl, n-pentyl, n-hexyl, n-heptyl, cyclopropyl, cyclobutyl, cyclopentyl, and cyclohexyl.

The expression ‘aryl’ means a phenyl, biphenyl or naphthyl group which optionally carries one or more substituents, preferably one or two substituents, each independently selected from cyano, halogen, lower alkyl, lower alkyloxy, lower alkenyloxy, trifluoromethyl, trifluoromethoxy, amino, carboxy and the like.

Preferred examples of aryl groups are phenyl, 4-methylphenyl, 4-methoxyphenyl, 4-bromophenyl, 4-cyanophenyl, 4-chlorophenyl, 4-fluorophenyl, 4-biphenyl, 2-methylphenyl, 2-methoxyphenyl, 2-bromophenyl, 2-cyanophenyl, 2-chlorophenyl, 2-fluorophenyl, 2-biphenyl, 3-methylphenyl, 3-methoxyphenyl, 3-bromophenyl, 3-cyanophenyl, 3-chlorophenyl, 3-fluorophenyl, 3-biphenyl, naphthalen-1-yl, and naphthalen-2-yl.

The expression ‘aryl-lower alkyl’ means a lower alkyl group as previously defined in which one hydrogen atom has been replaced by an aryl group as previously defined. Preferred examples of aryl-lower alkyl groups are 3-phenylpropyl, phenethyl, benzyl and benzyl substituted in the phenyl ring with hydroxy, lower alkyl, lower alkyloxy, or halogen.

Preferred examples of ‘(E)-2-aryl-ethen-1-yl’ groups are (E)-2-phenylethen-1-yl, (E)-2-(4-fluorophenyl)ethen-1-yl and (E)-3-phenylpropen-1-yl.

Preferred examples of ‘lower alkyl disubstituted with aryl’ groups are 2,2-diphenylethyl, 3,3-diphenylpropyl and 1-benzyl-2-phenyl-ethyl.

Preferred examples of ‘lower alkyl disubstituted with aryl and additionally substituted at a carbon atom bearing an aryl group with OH, CN or CONR⁷R⁸’ groups are 2,2-diphenyl-2-hydroxy-ethyl, N,N-dimethyl-2,2-diphenyl-4-yl-butyramide and N,N-diethyl-2,2-diphenyl-4-yl-butyramide.

The present invention encompasses pharmaceutically acceptable salts of compounds of the general formula 1. This encompasses either salts with inorganic acids or organic acids like hydrohalogenic acids, e.g. hydrochloric or hydrobromic acid, sulfuric acid, phosphoric acid, nitric acid, citric acid, formic acid, acetic acid, maleic acid, tartaric acid, methylsulfonic acid, p-tolylsulfonic acid and the like or in case the compound of formula 1 is acidic in nature with an inorganic base like an alkali or earth alkali base, e.g. sodium, potassium, or calcium salts, etc. The compounds of general formula 1 can also be present in form of zwitterions.

The present invention encompasses different solvation complexes of compounds of general formula 1. The solvation can be effected in the course of the manufacturing process or can take place separately, e.g. as a consequence of hygroscopic properties of an initially anhydrous compound of general formula 1.

The present invention further encompasses different morphological forms, e.g. crystalline forms, of compounds of general formula 1 and their salts and solvation complexes. Particular heteromorphs may exhibit different dissolution properties, stability profiles, and the like, and are all included in the scope of the present invention.

The compounds of the general formula 1 might have one or more asymmetric carbon atoms and may be prepared in form of optically pure enantiomers or diastereomers, mixtures of enantiomers or diastereomers, diastereomeric racemates, and mixtures of diastereomeric racemates. The present invention encompasses all these forms. They are prepared by stereoselective synthesis, or by separation of mixtures in a manner known per se, i.e. by column chromatography, thin layer chromatography, HPLC, crystallization, etc.

Preferred compounds of general formula 1 are the compounds wherein R³ forms together with R² an unsubstituted five-, six-, or seven-membered ring containing the nitrogen atom to which R² is attached as a ring atom, R⁴ is hydrogen and Py, X, and R¹ have the meaning given in general formula 1 above.

Another group of preferred compounds of general formula 1 consists of those compounds wherein R⁴ forms together with R² an unsubstituted five-, six-, or seven-membered ring containing the nitrogen atom to which R² is attached as a ring atom, R³ is hydrogen and Py, X, and R¹ have the meaning given in general formula 1 above.

Another group of preferred compounds of general formula 1 consists of those compounds wherein Py represents quinolin-4-yl mono- or disubstituted independently with lower alkyl or aryl-lower alkyl in the positions 2 or 8, and R¹, R², R³, R⁴, and X have the meaning given in general formula 1 above.

Another group of preferred compounds of general formula 1 consists of those compounds wherein Py represents pyridin-4-yl substituted in position 2 with R⁵R⁶N—, wherein R⁵ represents lower alkyl and R⁶ represents aryl-lower alkyl, and R¹, R², R³, R⁴, and X have the meaning given in general formula 1 above.

Another group of preferred compounds of general formula 1 consists of those compounds wherein Py represents pyridin-4-yl substituted in position 2 with R⁵R⁶N—, wherein R⁶ represents hydrogen and R¹, R², R³, R⁴, R⁵, and X have the meaning given in general formula 1 above.

Another group of preferred compounds of general formula 1 consists of those compounds wherein X is absent and R¹, R², R³, R⁴, and Py have the meaning given in general formula 1 above.

Another group of preferred compounds of general formula 1 consists of those compounds wherein Py represents pyridin-4-yl disubstituted in position 2 and 6 with lower-alkyl, and R¹, R², R³, R⁴, and X have the meaning given in general formula 1 above.

Another group of preferred compounds of general formula 1 consists of those compounds wherein Py represents pyridin-4-yl disubstituted in position 2 with aryl-lower alkyl and in position 6 with lower-alkyl, and R¹, R², R³, R⁴, and X have the meaning given in general formula 1 above.

Another group of preferred compounds of general formula 1 consists of those compounds wherein R¹ represents lower alkyl disubstituted with aryl and R², R³, R⁴, X, and Py have the meaning given in general formula 1 above.

Another group of preferred compounds of general formula 1 consists of those compounds wherein R¹ represents lower alkyl disubstituted with aryl and additionally substituted at a carbon atom bearing an aryl group with OH, CN, or CONR⁷R⁸, and R², R³, R⁴, R⁷, R⁸, X, and Py have the meaning given in general formula 1 above.

A group of especially preferred compounds of general formula 1 consists of those compounds wherein X is absent, R³ forms together with R² an unsubstituted five-, six-, or seven-membered ring containing the nitrogen atom to which R² is attached as a ring atom, R⁴is hydrogen, Py represents quinolin-4-yl mono- or disubstituted independently with lower alkyl or aryl-lower alkyl in the positions 2 or 8, and R¹ has the meaning given in general formula 1 above.

Another group of especially preferred compounds of general formula 1 consists of those compounds wherein X is absent, R³ forms together with R² an unsubstituted five-, six-, or seven-membered ring containing the nitrogen atom to which R² is attached as a ring atom, R⁴is hydrogen, Py represents pyridin-4-yl substituted in position 2 with R⁵R⁶N—, wherein R⁶ represents aryl-lower alkyl and R⁵ represents lower alkyl, and R¹ has the meaning given in general formula 1 above.

Another group of especially preferred compounds of general formula 1 consists of those compounds wherein X is absent, R³ forms together with R² an unsubstituted five-, six-, or seven-membered ring containing the nitrogen atom to which R² is attached as a ring atom, R⁴is hydrogen, Py represents pyridin-4-yl substituted in position 2 with R⁵R⁶N—, wherein R⁶ represents hydrogen, and R¹, and R⁵ have the meaning given in general formula 1 above.

Another group of especially preferred compounds of general formula 1 consists of those compounds wherein X is absent, R³ forms together with R² an unsubstituted five-, six-, or seven-membered ring containing the nitrogen atom to which R² is attached as a ring atom, R⁴is hydrogen, Py represents pyridin-4-yl disubstituted in position 2 and 6 with lower-alkyl, and R¹ has the meaning given in general formula 1 above.

Another group of especially preferred compounds of general formula 1 consists of those compounds wherein X is absent, R³ forms together with R² an unsubstituted five-, six-, or seven-membered ring containing the nitrogen atom to which R² is attached as a ring atom, R⁴ is hydrogen, Py represents pyridin-4-yl disubstituted in position 2 with aryl-lower alkyl and in position 6 with lower-alkyl, and R¹ has the meaning given in general formula 1 above.

Another group of especially preferred compounds of general formula 1 consists of those compounds wherein X is absent, R³ forms together with R² an unsubstituted five-, six-, or seven-membered ring containing the nitrogen atom to which R² is attached as a ring atom, R⁴ is hydrogen, R¹ represents lower alkyl disubstituted with aryl, and Py has the meaning given in general formula 1 above.

A group of most preferred compounds of general formula 1 consists of those compounds wherein X is absent, R³ forms together with R² an unsubstituted five-membered ring containing the nitrogen atom to which R² is attached as a ring atom, R⁴ is hydrogen, Py represents quinolin-4-yl monosubstituted with lower alkyl or aryl-lower alkyl in the position 2 and R¹ has the meaning given in general formula 1 above.

Another group of most preferred compounds of general formula 1 consists of those compounds wherein X is absent, R³ forms together with R² an unsubstituted five-membered ring containing the nitrogen atom to which R² is attached as a ring atom, R⁴ is hydrogen, Py represents pyridin-4-yl substituted in position 2 with R⁵R⁶N—, wherein R⁶ represents hydrogen and R¹, and R⁵ have the meaning given in general formula 1 above.

Another group of most preferred compounds of general formula 1 consists of those compounds wherein X is absent, R³ forms together with R² an unsubstituted five-membered ring containing the nitrogen atom to which R² is attached as a ring atom, R⁴ is hydrogen, Py represents pyridin-4-yl disubstituted in position 2 and 6 with lower-alkyl and R¹ has the meaning given in general formula 1 above.

Another group of most preferred compounds of general formula 1 consists of those compounds wherein X is absent, R³ forms together with R² an unsubstituted five-membered ring containing the nitrogen atom to which R² is attached as a ring atom, R⁴ is hydrogen, R¹ represents lower alkyl disubstituted with aryl, and Py has the meaning given in general formula 1 above.

Examples of particularly preferred compounds of general formula 1 are: 1-(2-Methyl-quinolin-4-yl)-3-pyrrolidin-3-yl-urea; 1-[1-(2,2-Diphenyl-ethyl)-pyrrolidin-3-yl]-3-(2-methyl-quinolin-4-yl)-urea; 1-[1-(1-Benzyl-2-phenyl-ethyl)-pyrrolidin-3-yl]-3-(2-methyl-quinolin-4-yl)-urea; 1-(2-Methyl-quinolin-4-yl)-3-(1-phenethyl-pyrrolidin-3-yl)-urea; 1-(2-Methyl-quinolin-4-yl)-3-[1-(3-phenyl-propyl)-pyrrolidin-3-yl]-urea; 1-(2-Methyl-quinolin-4-yl)-3-(1-naphthalen-1-ylmethyl-pyrrolidin-3-yl)-urea; 1-(2-Methyl-quinolin-4-yl)-3-(1-naphthalen-2-ylmethyl-pyrrolidin-3-yl)-urea; 1-(1-Biphenyl-4-ylmethyl-pyrrolidin-3-yl)-3-(2-methyl-quinolin-4-yl)-urea; 1-(2-Methyl-quinolin-4-yl)-3-[1-(4-phenyl-cyclohexyl)-pyrrolidin-3-yl]-urea; 1-[(R)-1-(1-Methyl-2,2-diphenyl-ethyl)-pyrrolidin-3-yl]-3-(2-methyl-quinolin-4-yl)-urea; 1-[(S)-1-(1-Methyl-2,2-diphenyl-ethyl)-pyrrolidin-3-yl]-3-(2-methyl-quinolin-4-yl)-urea; 1-[1-(3,3-Diphenyl-propyl)-pyrrolidin-3-yl]-3-(2-methyl-quinolin-4-yl)-urea; 1-[1-(2,3-Diphenyl-propyl)-pyrrolidin-3-yl]-3-(2-methyl-quinolin-4-yl)-urea; 1-[1-(2-Hydroxy-2,2-diphenyl-ethyl)-pyrrolidin-3-yl]-3-(2-methyl-quinolin-4-yl)-urea; 1-[1-(2,2-Diphenyl-ethyl)-piperidin-3-yl]-3-(2-methyl-quinolin-4-yl)-urea; 1-[1-(3,3-Diphenyl-propyl)-piperidin-3-yl]-3-(2-methyl-quinolin-4-yl)-urea; 1-[(S)-1-(1-Benzyl-2-phenyl-ethyl)-pyrrolidin-3-yl]-3-(2-methyl-quinolin-4-yl)-urea; 1-[(R)-1-(1-Benzyl-2-phenyl-ethyl)-pyrrolidin-3-yl]-3-(2-methyl-quinolin-4-yl)-urea; 1-[(S)-1-(3,3-Diphenyl-propyl)-pyrrolidin-3-yl]-3-(2-methyl-quinolin-4-yl)-urea; 1-[(R)-1-(3,3-Diphenyl-propyl)-pyrrolidin-3-yl]-3-(2-methyl-quinolin-4-yl)-urea; (R)-1-(1-Benzyl-pyrrolidin-3-yl)-3-(2-methyl-quinolin-4-yl)-urea; (S)-1-(1-Benzyl-pyrrolidin-3-yl)-3-(2-methyl-quinolin-4-yl)-urea; 1-(1-Benzyl-pyrrolidin-3-yl)-3-(2-methyl-quinolin-4-yl)-urea; 1-[(S)-1-(2-Hydroxy-2,2-diphenyl-ethyl)-pyrrolidin-3-yl]-3-(2-methyl-quinolin-4-yl)-urea; 1-[(R)-1-(2-Hydroxy-2,2-diphenyl-ethyl)-pyrrolidin-3-yl]-3-(2-methyl-quinolin-4-yl)-urea; 1-[(S)-1-(1-Benzyl-2-phenyl-ethyl)-pyrrolidin-2-ylmethyl]-3-(2-methyl-quinolin-4-yl)-urea; 1-[(R)-1-(1-Benzyl-2-phenyl-ethyl)-pyrrolidin-2-ylmethyl]-3-(2-methyl-quinolin-4-yl)-urea; N,N-Diethyl-4-{(S)-3-[3-(2-methyl-quinolin-4-yl)-ureido]-pyrrolidin-1-yl}-2,2-diphenyl-butyramide; N,N-Diethyl-4-{(R)-3-[3-(2-methyl-quinolin-4-yl)-ureido]-pyrrolidin-1-yl}-2,2-diphenyl-butyramide; N,N-Dimethyl-4-{(S)-3-[3-(2-methyl-quinolin-4-yl)-ureido]-pyrrolidin-1-yl}-2,2-diphenyl-butyramide; N,N-Dimethyl-4-{(R)-3-[3-(2-methyl-quinolin-4-yl)-ureido]-pyrrolidin-1-yl}-2,2-diphenyl-butyramide; 1-(1-Biphenyl-3-ylmethyl-pyrrolidin-3-yl)-3-(2-methyl-quinolin-4-yl)-urea; 1-((S)-1-Biphenyl-2-ylmethyl-pyrrolidin-3-yl)-3-(2-methyl-quinolin-4-yl)-urea; 1-[(S)-1-(3-Cyano-3,3-diphenyl-propyl)-pyrrolidin-3-yl]-3-(2-methyl-quinolin-4-yl)-urea; 1-[(R)-1-(3-Cyano-3,3-diphenyl-propyl)-pyrrolidin-3-yl]-3-(2-methyl-quinolin-4-yl)-urea; 1-[(S)-1-(1-Benzyl-2-phenyl-ethyl)-pyrrolidin-3-yl]-3-(2,6-dimethyl-pyridin-4-yl)-urea; 1-[(R)-1-(1-Benzyl-2-phenyl-ethyl)-pyrrolidin-3-yl]-3-(2,6-dimethyl-pyridin-4-yl)-urea; 1-(2,6-Dimethyl-pyridin-4-yl)-3-[(S)-1-(2,2-diphenyl-ethyl)-pyrrolidin-3-yl]-urea; 1-(2,6-Dimethyl-pyridin-4-yl)-3-[(S)-1-(2-hydroxy-2,2-diphenyl-ethyl)-pyrrolidin-3-yl]-urea; 1-(2,6-Dimethyl-pyridin-4-yl)-3-[(R)-1-(2-hydroxy-2,2-diphenyl-ethyl)-pyrrolidin-3-yl]-urea; 1-(2,6-Dimethyl-pyridin-4-yl)-3-[(S)-1-(3,3-diphenyl-propyl)-pyrrolidin-3-yl]-urea; 1-(2,6-Dimethyl-pyridin-4-yl)-3-[(R)-1-(3,3-diphenyl-propyl)-pyrrolidin-3-yl]-urea; 1-[(S)-1-(1-Benzyl-2-phenyl-ethyl)-pyrrolidin-3-yl]-3-(2-ethyl-6-methyl-pyridin-4-yl)-urea; 1-[(S)-1-(2,2-Diphenyl-ethyl)-pyrrolidin-3-yl]-3-(2-ethyl-6-methyl-pyridin-4-yl)-urea; 1-[(S)-1-(3,3-Diphenyl-propyl)-pyrrolidin-3-yl]-3-(2-ethyl-6-methyl-pyridin-4-yl)-urea; 1-[(S)-1-(3,3-Diphenyl-propyl)-pyrrolidin-3-yl]-3-[2-methyl-6-((E)-styryl)-pyridin-4-yl]-urea; 1-[(S)-1-(2,2-Diphenyl-ethyl)-pyrrolidin-3-yl]-3-{2-[(E)-2-(4-fluoro-phenyl)-vinyl]-6-methyl-pyridin-4-yl}-urea; 1-[(S)-1-(2,2-Diphenyl-ethyl)-pyrrolidin-3-yl]-3-(2-methyl-6-phenethyl-pyridin-4-yl)-urea; 1-[(S)-1-(1-Benzyl-2-phenyl-ethyl)-pyrrolidin-3-yl]-3-(2-methyl-6-propyl-pyridin-4-yl)-urea; 1-[(S)-1-(2,2-Diphenyl-ethyl)-pyrrolidin-3-yl]-3-(2-methyl-6-propyl-pyridin-4-yl)-urea; 1-[(S)-1-(3,3-Diphenyl-propyl)-pyrrolidin-3-yl]-3-(2-methyl-6-propyl-pyridin-4-yl)-urea; 1-[2-(Benzyl-methyl-amino)-pyridin-4-yl]-3-[(S)-1-(2,2-diphenyl-ethyl)-pyrrolidin-3-yl]-urea; 1-[(S)-1-(3,3-Diphenyl-propyl)-pyrrolidin-3-yl]-3-(2-methyl-6-phenethyl-pyridin-4-yl)-urea; 1-[(S)-1-(2,2-Diphenyl-ethyl)-pyrrolidin-3-yl]-3-{2-[2-(4-fluoro-phenyl)-ethyl]-6-methyl-pyridin-4-yl}-urea; 1-[(S)-1-(2,2-Diphenyl-ethyl)-pyrrolidin-3-yl]-3-(2-methylamino-pyridin-4-yl)-urea; 1-[(S)-1-(2,2-Diphenyl-ethyl)-pyrrolidin-3-yl]-3-(2-propylamino-pyridin-4-yl)-urea; 1-(2-Cyclopentylamino-pyridin-4-yl)-3-[(S)-1-(2,2-diphenyl-ethyl)-pyrrolidin-3-yl]-urea; 1-(2-Benzylamino-pyridin-4-yl)-3-[(S)-1-(2,2-diphenyl-ethyl)-pyrrolidin-3-yl]-urea.

Because of their ability to inhibit the actions of urotensin II, the described compounds can be used for treatment of diseases which are associated with an increase in vasoconstriction, proliferation or other disease states associated with the actions of urotensin II. Examples of such diseases are hypertension, atherosclerosis, angina or myocardial ischemia, congestive heart failure, cardiac insufficiency, cardiac arrhythmias, renal ischemia, chronic kidney disease, renal failure, stroke, cerebral vasospasm, cerebral ischemia, dementia, migraine, subarachnoidal hemorrhage, diabetes, diabetic arteriopathy, diabetic nephropathy, connective tissue diseases, cirrhosis, asthma, chronic obstructive pulmonary disease, high-altitude pulmonary edema, Raynaud's syndrome, portal hypertension, thyroid dysfunction, pulmonary edema, pulmonary hypertension, or pulmonary fibrosis. They can also be used for prevention of restenosis after balloon or stent angioplasty, for the treatment of cancer, prostatic hypertrophy, erectile dysfunction, hearing loss, amaurosis, chronic bronchitis, asthma, gram negative septicemia, shock, sickle cell anemia, glomerulonephritis, renal colic, glaucoma, therapy and prophylaxis of diabetic complications, complications of vascular or cardiac surgery or after organ transplantation, complications of cyclosporin treatment, pain, addictions, schizophrenia, Alzheimer's disease, anxiety, obsessive-compulsive behavior, epileptic seizures, stress, depression, dementias, neuromuscular disorders, neurodegenerative diseases, as well as other diseases related to a dysregulation of urotensin II or urotensin II receptors.

These compositions may be administered in enteral or oral form e.g. as tablets, dragees, gelatine capsules, emulsions, solutions or suspensions, in nasal form like sprays or rectally in form of suppositories. These compounds may also be administered in intramuscular, parenteral or intravenous form, e.g. in form of injectable solutions.

These pharmaceutical compositions may contain the compounds of formula 1 as well as their pharmaceutically acceptable salts in combination with inorganic and/or organic excipients, which are usual in the pharmaceutical industry, like lactose, maize or derivatives thereof, talcum, stearic acid or salts of these materials.

For gelatine capsules vegetable oils, waxes, fats, liquid or half-liquid polyols etc. may be used. For the preparation of solutions and sirups e.g. water, polyols, saccharose, glucose etc. are used. Injectables are prepared by using e.g. water, polyols, alcohols, glycerin, vegetable oils, lecithin, liposomes etc. Suppositories are prepared by using natural or hydrogenated oils, waxes, fatty acids (fats), liquid or half-liquid polyols etc.

The compositions may contain in addition preservatives, stabilisation improving substances, viscosity improving or regulating substances, solubility improving substances, sweeteners, dyes, taste improving compounds, salts to change the osmotic pressure, buffer, anti-oxidants etc.

The compounds of general formula 1 may also be used in combination with one or more other therapeutically useful substances e.g. α- and β-blockers like phentolamine, phenoxybenzamine, atenolol, propranolol, timolol, metoprolol, carteolol, carvedilol, etc.; with vasodilators like hydralazine, minoxidil, diazoxide, flosequinan, etc.; with calcium-antagonists like diltiazem, nicardipine, nimodipine, verapamil, nifedipine, etc.; with angiotensin converting enzyme-inhibitors like cilazapril, captopril, enalapril, lisinopril etc.; with potassium channel activators like pinacidil, chromakalim, etc.; with angiotensin receptor antagonists like losartan, valsartan, candesartan, irbesartan, eprosartan, telmisartan, and tasosartan, etc.; with diuretics like hydrochlorothiazide, chlorothiazide, acetolamide, bumetanide, furosemide, metolazone, chlortalidone, etc.; with sympatholytics like methyldopa, clonidine, guanabenz, reserpine, etc.; with endothelin receptor antagonists like bosentan, tezosentan, darusentan, atrasentan, enrasentan, or sitaxsentan, etc.; with anti-hyperlipidemic agents like lovastatin, pravistatin, fluvastatin, atorvastatin, cerivastatin, simvastatin, etc.; and other therapeutics which serve to treat high blood pressure, vascular disease or other disorders listed above.

The dosage may vary within wide limits but should be adapted to the specific situation. In general the dosage given daily in oral form should be between about 3 mg and about 3 g, preferably between about 5 mg and about 1 g, especially preferred between 10 mg and 300 mg, per adult with a body weight of about 70 kg. The dosage should be administered preferably in 1 to 3 doses of equal weight per day. As usual children should receive lower doses which are adapted to body weight and age.

### GENERAL PREPARATION OF COMPOUNDS OF THE INVENTION

Compounds of the general formula 1 can be prepared using methods generally known in the art, according to the general sequence of reactions outlined below. For simplicity and clarity reasons sometimes only a few of the possible synthetic routes that lead to compounds of general formula 1 are described.

For the synthesis of compounds of general formula 1 general synthetic routes illustrated in Schemes A through G can be employed. The generic groups X, Py, R², R¹, R³, R⁴, R⁵, R⁶, R⁷, R⁸ employed in Schemes A through G have the definitions given in general formula 1 above. In some instances the use of protecting groups (PG) will be required. The use of protecting groups is well known in the art (see for example “Protective Groups in Organic Synthesis, T. W. Greene, P. G. M. Wuts, Wiley-Interscience, 1999). For the purposes of this discussion, it will be assumed that protecting groups such as benzyloxycarbonyl (Cbz), benzyl (Bn) or tert-butyloxycarbonyl (Boc) are in place.

Preparation of Compounds of General Formula 1.

These compounds are prepared according to Scheme A.

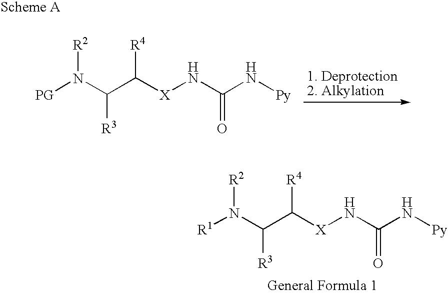

1,3-Disubstituted ureas of general structure I in Scheme A are deprotected at the nitrogen attached to R² according to procedures well known in the art (see for example “Protective Groups in Organic Synthesis, T. W. Greene, P. G. M. Wuts, Wiley-Interscience, 1999) and subsequently alkylated to provide compounds of general formula 1. N-Alkylation is preferentially accomplished by reductive amination, using NaBHAc₃ as reducing agent in THF, with aldehydes or ketones that are commercially available or are prepared by methods well-known in the art. Alternatively, N-alkylation can be accomplished, in a polar solvent such as THF in the presence of a small stoichiometric excess of acid scavenger such as Na₂CO₃ or DIPEA, by reaction with halides R¹—X or methanesulfonates R¹—OSO₂CH₃ that are commercially available or are prepared by methods well-known in the art. Alternatively, N-alkylation can be accomplished, in a polar solvent such as THF in the presence of a small stoichiometric excess of acid scavenger such as TEA or DIPEA, by reaction with activated carboxylic acid derivatives that are commercially available or are prepared by methods well-known in the art, followed by reduction of the amide intermediate by treatment with a reducing agent such as LiAlH₄ in an aprotic solvent such as THF at room temperature. The preparation of protected ureas of general structure I is described in Schemes D to F below.

Alternatively, compounds of general formula 1 are prepared according to Scheme B and C.

Racemic or enantiomerically pure amines of general structure IV are either commercially available or readily prepared by methods well known in the art. Pyridine-4-carboxylic acid derivatives of general structure II are commercially available or readily prepared by methods well known in the art. According to Scheme B amines of general structure IV are reacted in a solvent such as CH₂Cl₂ with isocyanates, formed in situ from acids of general structure II via rearrangement of the derived acyl azides, to provide protected ureas of general structure I. Alternatively, ureas of general structure I can be formed by reaction of an amine of general structure IV and an urea of general structure III by heating in a polar solvent such as dioxane or methanol as shown in Scheme C. Ureas of general structure III are prepared according to Scheme G below.

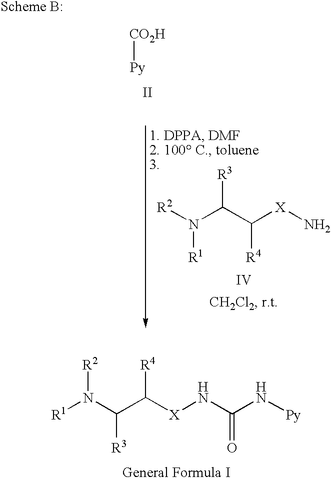

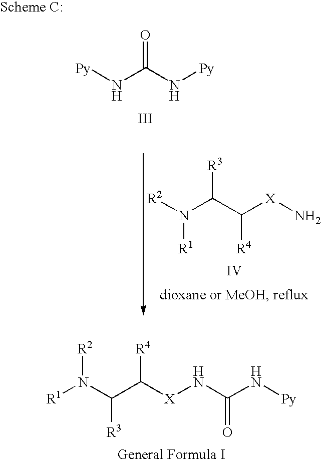

Protected ureas of general structure I in Scheme A are prepared according to Scheme D below.

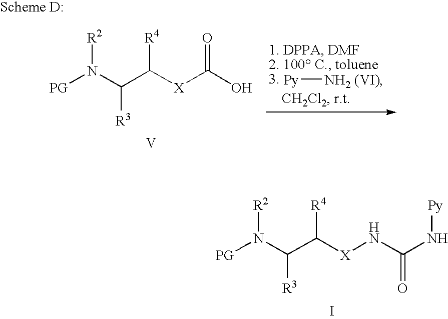

Monoprotected, racemic or enantiomerically pure carboxylic acids of general structure V are either commercially available or readily prepared by methods well known in the art. 4-Amino-pyridine derivatives of general structure VI are commercially available or readily prepared by methods well known in the art (see for example “A Convenient Preparation of 4-Pyridinamine Derivatives, M. Malinowski, L. Kaczmarek, J. Prakt. Chem. (1988) 330, 154-158). According to Scheme D 4-amino-pyridine derivatives of general structure VI are reacted in a solvent such as CH₂Cl₂ with isocyanates, formed in situ from acids of general structure V via rearrangement of the derived acyl azides, to provide protected ureas of general structure I.

Alternatively, protected ureas of general structure I in Scheme A are prepared according to Schemes E and F below.

Monoprotected, racemic or enantiomerically pure amines of general structure VII are either commercially available or readily prepared by methods well known in the art. According to Scheme E and F, using general methods described in Scheme B and C for the preparation of compounds of general formula 1, amines of general structure VII are reacted with isocyanates, formed in situ from acids of general structure II to provide protected ureas of general structure I. Alternatively, amines of general structure VII are reacted with an urea of general structure III to provide protected ureas of general structure I.

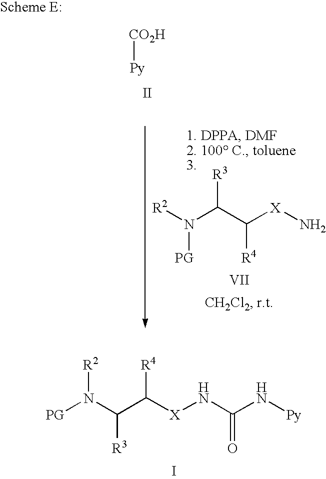

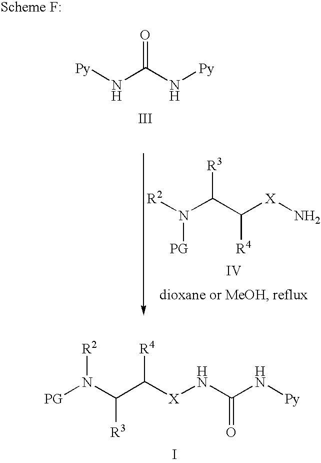

Ureas of general structure III are prepared according to Scheme G below.

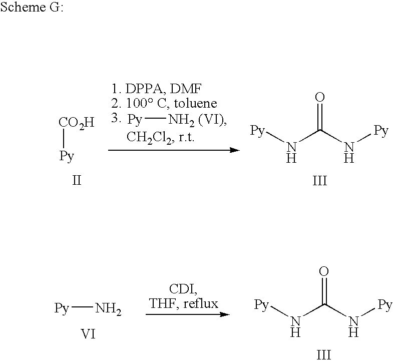

Pyridine-4-carboxylic acid derivatives of general structure II are commercially available or readily prepared by methods well known in the art. 4-Amino-pyridine derivatives of general structure VI are commercially available or readily prepared by methods well known in the art. According to Scheme G 4-amino-pyridine derivatives of general structure VI are reacted in a solvent such as CH₂Cl₂ with isocyanates, formed in situ from acids of general structure II via rearrangement of the derived acyl azides, to provide ureas of general structure III. Alternatively, 4-amino-pyridine derivatives of general structure VI are reacted in a polar, aprotic solvent such as THF with carbonyldiimidazole (CDI) to provide ureas of general structure III.

The foregoing general description of the invention will now be further illustrated with a number of non-limiting examples.

### EXAMPLES

### LIST OF ABBREVIATIONS

AcOH acetic acid
aq. aqueous
brine sat. sodium chloride solution in water
BSA bovine serum albumin
cat. catalytic
CDI carbonyldiimidazole
DIPEA diisopropylethylamine
DMAP 4-dimethylaminopyridine
DMF dimethylformamide
DMSO dimethylsulfoxide
DPPA diphenylphosphorylazide
EDC N-(3-dimethylaminopropyl)-N′-ethyl-carbodiimide
EDTA ethylenediamine tetra-acetic acid
EtOAc ethyl acetate
Et₂O diethyl ether
FC flash chromatography
Fe(acac)₃ iron(III)-acetylacetonate
Hex hexane
HOBt 1-hydroxybenzotriazole
HPLC high performance liquid chromatography
HV high vacuum conditions
LC-MS liquid chromatography-mass spectroscopy
LiAlH₄ lithium aluminum hydride
MeOH methanol
min minutes
MHz megahertz
MPLC medium pressure liquid chromatography
NaBHAC₃ sodium triacetoxyborohydride
NaHMDS sodium bis(trimethylsilyl)amide
NMP N-methylpyrrolidone
NMR nuclear magnetic resonance
ppm part per million
PBS phosphate-buffered saline
Pd(dppf)Cl₂ 1,1′-bis(diphenylphosphino)ferrocene-palladium(II)dichloride dichloromethane complex
PG protecting group
r.t. room temperature
sat. saturated
SiO₂ silica gel
TEA triethylamine
TFA trifluoroacetic acid
THF tetrahydrofuran
TLC thin layer chromatography
t_{R} retention time

Reactions are routinely performed under an inert atmosphere such as N₂ gas in air dried glassware. Solvents are used as received from the vendor. Evaporations are performed in a rotary evaporator at reduced pressure and a water bath temperature of 50° C. LC-MS characterizations are performed on a Finnigan HP1100 platform using ESI ionization mode, and positive ion detection with a Navigator AQA detector. Analytical liquid chromatographic separations are performed on a C18 column of 4.6×30 mm dimensions and a mobile phase consisting of a 6 minute gradient of 2-95% CH₃CN in water containing 0.5% formic acid at a flow rate of 0.45 mL/min. Retention time (t_{R}) is given in min. TLC is performed on pre-coated silica gel 60 F₂₅₄ glass-backed plates (Merck). MPLC is performed on a Labomatic platform using either SiO₂-columns and a mobile phase consisting of heptane-EtOAc, or C18 columns and a mobile phase consisting of water-MeOH. Preparative HPLC is performed on a Varian/Gilson platform using a C18 column of 21×60 mm dimensions and a mobile phase consisting of a gradient of 2-95% CH₃CN in water containing 0.5% formic acid.

### Preparation of Intermediates

### Example A

### A1. 1-Benzyl-pyrrolidin-3-ylamine

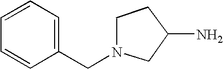

This material is commercially available in racemic and both enantiomerically pure forms.

### A2. 3-Amino-pyrrolidine-1-carboxylic acid tert-butyl ester

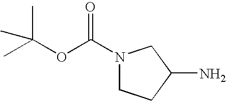

This material is commercially available in racemic form.

### A3. Pyrrolidin-3-yl-carbamic acid tert-butyl ester

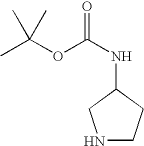

This material is commercially available in racemic and both enantiomerically pure forms. cl A4. 3-Amino-piperidine-1-carboxylic acid tert-butyl ester

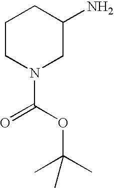

This material is commercially available in racemic form.

### A5. (S)-1-(2,2-Diphenyl-ethyl)-pyrrolidin-3-ylamine

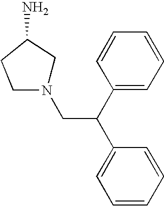

### A5.1. [(S)-1-(2,2-Diphenyl-ethyl)-pyrrolidin-3-yl-carbamic acid tert-butyl ester

A mixture of (S)-pyrrolidin-3-yl-carbamic acid tert-butyl ester (Example A3., 2.5 g, 13.4 mmol), diphenylacetaldehyde (2.63 g, 13.4 mmol) and NaBHAc3 (4.0 g, 19 mmol) in THF (80 mL) is stirred at r.t. for 6 h. The mixture is diluted with CH₂Cl₂ (150 mL) and washed with sat. aq. Na₂CO₃ (2×50 mL) and sat. aq. NaCl (50 mL). The organic phase is dried (Na₂SO₄), filtered and evaporated. The residue is purified by FC (SiO₂, EtOAc-heptane) to provide the title compound.

### A5.2. (S)-1-(2,2-Diphenyl-ethyl)-pyrrolidin-3-ylamine

To a solution of [(S)-1-(2,2-diphenyl-ethyl)-pyrrolidin-3-yl]-carbamic acid tert-butyl ester (4.37 g, 11.9 mmol) in CHCl₃ (50 mL) is added TFA (20 mL) and the mixture is stirred at r.t. for 2 h. The mixture is evaporated, the residue dissolved in CH₂Cl₂ (100 mL) and stirred with aq. NaOH (1M, 100 mL) for 1 h. The phases are separated and the aq. phase is extracted with CH₂Cl₂ (2×30 mL). The combined organic extracts are dried (Na₂SO₄), filtered and evaporated to provide the title compound.

The following compounds are prepared from the appropriate stereoisomer of pyrrolidin-3-yl-carbamic acid tert-butyl ester (Example A3) and commercially available aldehydes or ketones using the method described in Example A5.

| Example No | Example |
|---|---|
| A6. | (R)-1-(2,2-Diphenyl-ethyl)-pyrrolidin-3-ylamine |
| A7. | (S)-1-(1-Benzyl-2-phenyl-ethyl)-pyrrolidin-3-ylamine |
| A8. | (R)-1-(1-Benzyl-2-phenyl-ethyl)-pyrrolidin-3-ylamine |

### A9. (S)-1-(3,3-Diphenyl-propyl)-pyrrolidin-3-ylamine

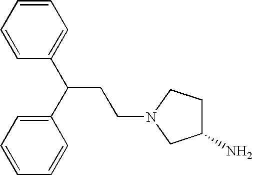

### A9.1. [(S)-1-(3,3-Diphenyl-propionyl)-pyrrolidin-3-yl-carbamic acid tert-butyl ester

To a cooled (0° C.) mixture of (S)-pyrrolidin-3-yl-carbamic acid tert-butyl ester (Example A3., 930 mg, 5 mmol), 3,3-diphenylpropionic acid (1.36 g, 6 mmol), HOBt (1.35 g, 10 mmol), TEA (1.4 mL, 10 mmol) and a cat. amount of DMAP in CH₂Cl₂ (50 mL) is added EDC (1.15 g, 6 mmol). The mixture is stirred at r.t. for 15 h. The mixture is quenched with sat. aq. Na₂CO₃ (25 mL), the phases are separated, and the aq. phase is extracted with CH₂Cl₂ (3×50 mL). The combined organic extracts are dried (Na₂SO₄), filtered and evaporated. The residue is purified by FC (SiO₂, EtOAc-heptane) to provide the crude title compound.

### A9.2. [(S)-1-(3,3-Diphenyl-propyl)-pyrrolidin-3-yl]-carbamic acid tert-butyl ester

A solution of [(S)-1-(3,3-diphenyl-propionyl)-pyrrolidin-3-yl]-carbamic acid tert-butyl ester (1.97 g, 5 mmol) in THF (20 mL) is added to a cooled (0° C.) suspension of LiAlH₄ (760 mg, 20 mmol) in THF (100 mL) and the mixture is warmed to r.t. during 15 h. The reaction mixture is carefully added to EtOAc (250 mL) and MeOH (30 mL), and, subsequently, sat. aq. NaHCO₃ (25 mL) are added until a filterable precipitate has formed. The mixture is filtered, the filtercake washed with MeOH (2×50 mL), and the filtrate is evaporated. The residue is taken up in a minimal amount of MeOH, diluted with CH₂Cl₂ (300 mL), dried (Na₂SO₄), filtered and evaporated. The residue is purified by FC (SiO₂, EtOAc-heptane) to provide the title compound.

### A9.3. (S)-1-(3,3-Diphenyl-propyl)-pyrrolidin-3-ylamine

To a solution of [(S)-1-(3,3-diphenyl-propyl)-pyrrolidin-3-yl]-carbamic acid tert-butyl ester (1.97 g, 5 mmol) in CHCl₃ (50 mL) is added TFA (20 mL) and the mixture is stirred at r.t. for 2 h. The mixture is evaporated, the residue dissolved in CH₂Cl₂ (100 mL) and stirred with aq. NaOH (1 M, 100 mL) for 1 h. The phases are separated and the aq. phase is extracted with CH₂Cl₂ (2×30 mL). The combined organic extracts are dried (Na₂SO₄), filtered and dried to provide the title compound.

The following compounds are prepared from the appropriate stereoisomer of pyrrolidin-3-yl-carbamic acid tert-butyl ester (Example A3) and commercially available carboxylic acids using the method described in Example A9.

| Example No | Example |
|---|---|
| A10. | (R)-1-(3,3-Diphenyl-propyl)-pyrrolidin-3-ylamine |
| A11. | 2-((S)-3-Amino-pyrrolidin-1-yl)-1,1-diphenyl-ethanol |
| A12. | 2-((R)-3-Amino-pyrrolidin-1-yl)-1,1-diphenyl-ethanol |

### A13. C-[(S)-1-(1-Benzyl-2-phenyl-ethyl)-pyrrolidin-2-yl]-methylamine

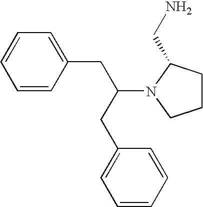

A mixture of L-prolinamide (121 mg, 1.06 mmol), dibenzylketone (223 mg, 1.06 mmol) and NaBHAc3 (270 mg, 1.27 mmol) in THF (4 mL) is stirred at r.t. for 15 h. The mixture is added to a cooled (0° C.) suspension of LiAlH₄ (224 mg, 5.3 mmol) in THF (15 mL) and the mixture is warmed to r.t. during 15 h. The reaction mixture is carefully added to EtOAc (100 mL) and MeOH (5 mL), and, subsequently, sat. aq. NaHCO₃ (2 mL) are added. The mixture is filtered, the filtercake washed with MeOH (2×20 mL), and the filtrate is evaporated. The residue is taken up in a minimal amount of MeOH, diluted with CH₂Cl₂ (100 mL), dried (Na₂SO₄), filtered and evaporated. The residue is purified by FC (SiO₂, EtOAc-MeOH) to provide the title compound.

### A14. C-[(R)-1-(1-Benzyl-2-phenyl-ethyl)-pyrrolidin-2-yl-methylamine

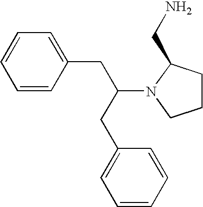

The compound is prepared from D-prolinamide and dibenzylketone using the method described in Example A13.

### A15. 4-((S)-3-Amino-pyrrolidin-1-yl)-N,N-diethyl-2.2-diphenyl-butyramide

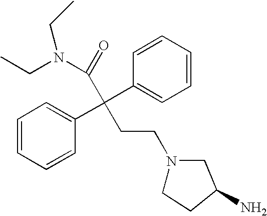

### A15.1. 4-Bromo-2,2-diphenyl-butyryl chloride

Thionylchloride (29 mL, 40 mmol) is added to a mixture of 4-bromo-2,2-diphenyl-butyric acid (3.05 g, 9.5 mmol) in CHCl₃ (50 mL) and the mixture is heated at reflux for 3 h. The mixture is evaporated in vacuo to provide the crude title compound.

### A15.2. [(S)-1-(3-Diethylcarbamoyl-3,3-diphenyl-propyl)-pyrrolidin-3-yl]-carbamic acid tert-butyl ester

A solution of 4-bromo-2,2-diphenyl-butyryl chloride (509 mg, 1.5 mmol) in CH₂Cl₂ (20 mL) is cooled at −10° C. and a solution of diethylamine (110 mg, 1.5 mmol) in CH₂Cl₂ (5 mL) is added, followed after 20 min by a solution of TEA (0.21 mL, 1.5 mmol) in CH₂Cl₂ (5 mL). The mixture is stirred for 10 min at −10° C. and a solution of (S)-pyrrolidin-3-yl-carbamic acid tert-butyl ester (186 mg, 1 mmol) in CH₂Cl₂ (5 mL) is added. The mixture is warmed to r.t. during 15 h, quenched with sat. aq. Na₂CO₃ (50 mL), the phases are separated and the aq. phase is extracted with CH₂Cl₂ (3×50 mL). The organic extracts are combined, dried (MgSO₄), filtered and evaporated. The residue is purified by MPLC (SiO₂, EtOAc-heptane) to provide the title compound.

### A15.3. 4-((S)-3-Amino-pyrrolidin-1-yl)-N,N-diethyl-2,2-diphenyl-butyramide

To a solution of [(S)-1-(3-diethylcarbamoyl-3,3-diphenyl-propyl)-pyrrolidin-3-yl]-carbamic acid tert-butyl ester (341 mg, 0.7 mmol) in CHCl₃ (10 mL) is added TFA (5 mL) and the mixture is stirred at r.t. for 0.5 h. The mixture is evaporated, the residue dissolved in CH₂Cl₂ (50 mL) and stirred with aq. NaOH (1 M, 30 mL) for 1 h. The phases are separated and the aq. phase is extracted with CH₂Cl₂ (2×30 mL). The combined organic extracts are dried (Na₂SO₄), filtered and dried to provide the title compound.

The following compounds are prepared from the appropriate stereoisomer of pyrrolidin-3-yl-carbamic acid tert-butyl ester (Example A3), 4-bromo-2,2-diphenyl-butyryl chloride (Example A15.1.) and commercially available dialkylamines using the method described in Example A15.

| Example No | Example |
|---|---|
| A16. | 4-((R)-3-Amino-pyrrolidin-1-yl)-N,N-diethyl-2,2-diphenyl- |
| | butyramide |
| A17. | 4-((S)-3-Amino-pyrrolidin-1-yl)-N,N-dimethyl-2,2- |
| | diphenyl-butyramide |
| A18. | 4-((R)-3-Amino-pyrrolidin-1-yl)-N,N-dimethyl-2,2- |
| | diphenyl-butyramide |

### Preparation of Intermediates

### Example B

### B1. 4-Amino-2-methylquinoline

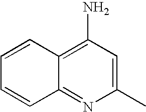

This material is commercially available.

### B2. 1,3-Bis-(2-methyl-quinolin-4-yl)-urea

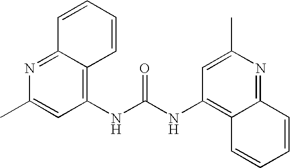

A suspension of 4-amino-2-methylquinoline (Example B1, 9.49 g, 60 mmol) and CDI (4.87 g, 20 mmol) in 100 ml THF is stirred at r.t. for 0.5 h, then 1 h at reflux. A second batch of CDI (2.5 g, 15.4 mmol) is added and heating continued for 15 h. The formed precipitate is filtered, washed with THF (2×50 mL) and ether (3×50 mL) and dried to provide the title compound.

### B3. 2,6-Dimethyl-pyridin-4-ylamine

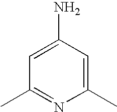

### B3.1. 2,6-Dimethyl-4-nitro-pyridine 1-oxide

Lutidine-N-oxide (19 g, 155 mmol) is cooled to 0° C. and a mixture of fuming HNO₃ (100 %, 37.5 mL) and conc. H₂SO₄ (95-97%, 52.5 mL), prepared by addition of H₂SO₄ to HNO₃ at 0° C., is added slowly. The mixture is heated at 80° C. for 3 h. The mixture is carefully poured into ice-water (500 mL). A white precipitate forms that is filtered. The precipitate is dissolved in CH₂Cl₂ (100 mL) and the filtrate is extracted with CH₂Cl₂ (4×75 mL). The organic extracts are combined with the dissolved precipitate and washed with sat. aq. NaCl, dried (Na₂SO₄), filtered and evaporated to provide the title compound.

### B3.2. 2,6-Dimethyl-pyridin-4-ylamine

2,6-Dimethyl-4-nitro-pyridine 1-oxide (9.62 g, 57 mmol) is dissolved in AcOH (300 mL) and Fe (29 g) is added. The mixture is stirred for 1 h at 100° C. The mixture is cooled to r.t. and filtered. The filtercake is thoroughly washed with AcOH and then discarded. The filtrate is evaporated, diluted with water (100 mL), basified with NaOH (1M, 100 mL), filtered from the formed precipitate and the filtrate is extracted with CHCl₃ (10×50 mL). The combined organic extracts are dried (Na₂SO₄), filtered and evaporated. The residue is crystallized from heptane-CHCl₃ to provide the title compound.

### B4. 1.3-Bis-(2.6-dimethyl-pyridin-4-yl)-urea

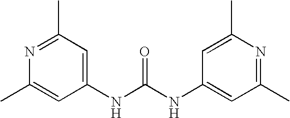

2,6-Dimethyl-pyridin-4-ylamine (1.22 g, 10 mmol) is dissolved in dry dioxane (30 mL) and CDI (891 mg, 5.5 mmol) is added. The mixture is heated at 80° C. for 1 h. Further CDI (160 mg) is added and stirring is continued for 15 h. The mixture is evaporated and purified by FC (SiO₂, EtOAc-MeOH) to provide the title compound.

### B5. 4-Isocyanato-2-methyl-6-styryl-pyridine

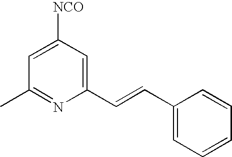

### B5.1. 2-Methyl-6-styryl-isonicotinic acid

A suspension of 2-chloro-6-methyl-isonicotinic acid (171.6 mg, 1 mmol), 2-phenyl-etheneboronic acid (180.0 mg, 1.2 mmol), K₂CO₃ (414 mg), Pd(dppf)Cl₂—CH₂Cl₂ (27 mg) in CH₃CN—H₂O (3:1, 10 mL) is stirred under argon at 90° C. for 15 h. The solution is cooled to r.t. and aq. hydrochloric acid (2M, 1.5 mL) is added to adjust the pH at 3. The mixture is evaporated to dryness and purified by MPLC (C18, H₂O-MeOH) to provide the title compound.

### B5.2. 2-Methyl-6-styryl-isonicotinoyl azide

To a solution of 2-methyl-6-styryl-isonicotinic acid (214 mg, 0.89 mmol) in DMF (5 mL) is added at 0° C. TEA (0.21 mL, 1.5 mmol) and slowly (30 min) DPPA (366 mg, 1.33 mmol). The reaction mixture is stirred for 0.5 h at 0° C. and 0.5 h at r.t. The reaction is quenched with ice (20 g) and extracted with Et₂O (6×30 mL). The combined organic extracts are washed successively with saturated NaHCO₃ (2×15 mL) and water (2×10 mL), and are evaporated in vacuo without heating.

The residue is purified by FC (SiO₂, EtOAc-heptane) to provide the title compound.

### B5.3. 4-Isocyanato-2-methyl-6-styryl-pyridine

2-Methyl-6-styryl-isonicotinoyl azide (79.9 mg, 0.3 mmol) is dissolved in dry toluene (4 mL) and heated at reflux for 2 h. The resulting solution of the title product is carried forward without further isolation of the title compound.

### B6. 2-[2-(4-Fluoro-phenyl)-vinyl]-4-isocyanato 6-methyl-pyridine

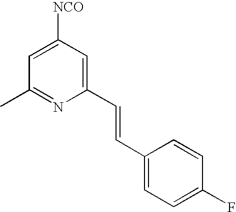

The title compound is prepared from 2-(4-fluoro-phenyl)-etheneboronic acid and 2-chloro-6-methyl-isonicotinic acid using the method described in Example B5.

### B7. 4-Isocyanato-2-methyl-6-phenethyl-pyridine

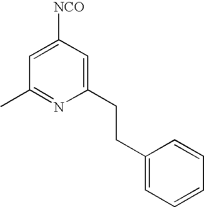

### B7.1. 2-Chloro-6-methyl-isonicotinic acid tert-butyl ester

N,N-dimethylformamide-di-tert.-butyl-acetal (19 mL, 80 mmol) is added during 40 min to a hot (65° C., flask temperature) suspension of 2-chloro-6-methyl-isonicotinic acid (3.40 g, 19.8 mmol) in dry toluene (100 mL). The clear orange solution is stirred at 80° C. for 48 h, cooled to r.t. and diluted with toluene (100 mL). The solution is washed with water (2×40 mL), sat. aq. NaHCO₃ (3×30 mL) and sat. aq. NaCl (25 mL), dried (Na₂SO₄), filtered and evaporated. The residue is purified by FC (SiO₂, CH₂Cl₂-MeOH) to provide the title compound.

### B7.2. 2-Methyl-6-phenethyl-isonicotinic acid

A solution of phenethylmagnesiumbromide (freshly prepared from phenethylbromide (0.66 g, 3.6 mmol) and magnesium (0.083 g, 3.4 mmol)) in ether (10 mL) is added to a cooled (−40° C.) and mechanically stirred solution of 2-chloro-6-methyl-isonicotinic acid tert-butyl ester (Example B7.1, 0.76 g, 3.34 mmol), Fe(acac)₃ (21.2 mg, 0.06 mmol) and NMP (0.6 mL) in THF (60 mL). The mixture is warmed to r.t. during 0.5 h, diluted with ether (150 mL) and quenched with aq. KHSO₄ (1M, 40 mL). The phases are separated and the aq. phase is extracted with ether (2×50 mL). The combined organic extracts are dried (MgSO₄), filtered and evaporated. The residue is purified by MPLC (C18, MeOH—H₂O) and the 2-methyl-6-phenethyl-isonicotinic acid tert-butyl ester dissolved in CH₂Cl₂ (10 mL). TFA (10 mL) is added and the mixture stirred at r.t. for 0.5 h. The mixture is evaporated and the residue dried in HV to provide the title compound.

### B7.3. 2-Methyl-6-phenethyl-isonicotinoyl azide

The title compound is prepared from 2-methyl-6-phenethyl-isonicotinic acid using the method described in Example B5.2.

### B7.4. 4-Isocyanato-2-methyl-6-phenethyl-pyridine

The title compound is prepared from 2-methyl-6-phenethyl-isonicotinoyl azide using the method described in Example B5.3.

### B8. 2-Ethyl-4-isocyanato-6-methyl-pyridine

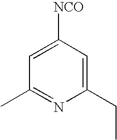

The title compound is prepared from 2-chloro-6-methyl-isonicotinic acid tert-butyl ester (Example B7.1.) and ethylbromide using the method described in Example B7.

### B9. 4-Isocyanato-2-methyl-6-propyl-pyridine

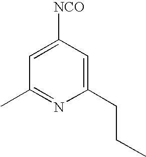

The title compound is prepared from 2-chloro-6-methyl-isonicotinic acid tert-butyl ester (Example B7.1.) and propylbromide using the method described in Example B7.

### B10. Benzyl-(4-isocyanato-pyridin-2-yl)-methyl-amine

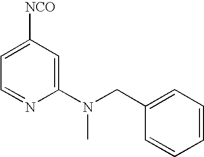

### B10.1. 2-(Benzyl-methyl-amino)-isonicotinic acid

A mixture of 2-chloro-pyridine-4-carboxylic acid (300 mg, 1.9 mmol), benzylmethylamine (230 mg, 1.9 mmol) and triethylamine (192 mg, 1.9 mmol) is heated at 120° C. for 12 h. The residue is dissolved in CH₂Cl₂ (30 mL) and extracted with 1 M aq. NaOH (3×5 mL). The aq. layer is adjusted to pH 1-2 with 12N aq. HCl and extracted with EtOAc (6×5 mL). The organic extracts are combined, dried (MgSO₄), and evaporated to provide the title compound.

### B10.2. 2-(Benzyl-methyl-amino)-isonicotinoyl azide

The title compound is prepared from 2-methyl-6-phenethyl-isonicotinic acid using the method described in Example B5.2.

### B10.3. Benzyl-(4-isocyanato-pyridin-2-yl)-methyl-amine

The title compound is prepared from 2-methyl-6-phenethyl-isonicotinoyl azide using the method described in Example B5.3.

### B11. (6-Chloro-4-isocyanato-pyridin-2-yl)-propyl-amine

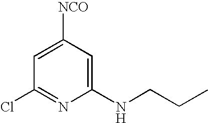

### B11.1. 2-Chloro-6-propylamino-isonicotinic acid

A mixture of n-propylamine (590 mg, 10 mmol) and 2,6-dichloroisonicotinic acid (192 mg, 1 mmol) is heated in a screw cap vial at 110° C. for 48 h. The excess amine is evaporated and the mixture is poured into 2M aq. HCl (30 mL) and washed with CH₂Cl₂ (3×30 mL), the organic extracts are combined, dried (Na₂SO₄), filtered and evaporated. The residue is suspended in MeOH (1 mL) and diluted with 1M aq. HCl (10 mL). The suspension is heated at 60° C. and the formed precipitate is filtered, washed with HCl (10 mL) and water (3×10 mL) and the solid is dried in HV to provide the title compound.

### B11.2. 2-Chloro-6-propylamino-isonicotinoyl azide

The title compound is prepared from 2-chloro-6-propylamino-isonicotinic acid using the method described in Example B5.2.

### B11.3. (6-Chloro-4-isocyanato-pyridin-2-yl)-propyl-amine

The title compound is prepared from 2-chloro-6-propylamino-isonicotinoyl azide using the method described in Example B5.3.

### B12. (6-Chloro-4-isocyanato-pyridin-2-yl)-cyclopentyl-amine

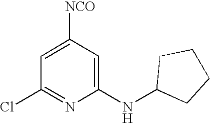

The title compound is prepared from cyclopentylamine and 2,6-dichloroisonicotinic acid using the method described in Example B11.

### B13. Benzyl-(6-chloro-4-isocyanato-pyridin-2-yl)-amine

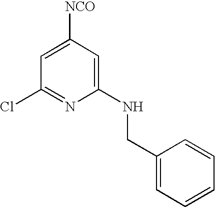

The title compound is prepared from benzylamine and 2,6-dichloroisonicotinic acid using the method described in Example B11.

### Preparation of Intermediates

### Example C

### C1. 1-(2-Methyl-quinolin-4-yl)-3-pyrrolidin-3-yl-urea

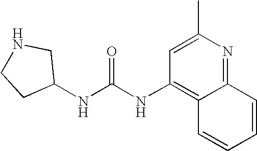

### C1.1. 3-[3-(2-Methyl-quinolin-4-yl)-ureido]-pyrrolidine-1-carboxylic acid tert-butyl ester

A suspension of 3-amino-pyrrolidine-1-carboxylic acid tert-butyl ester (Example A2, 820 mg, 4.4 mmol) and 1,3-bis-(2-methyl-quinolin-4-yl)-urea (Example B2, 1.51 g 4.4 mmol) in MeOH (20 mL) is heated at reflux for 15 h. The mixture is cooled to r.t. and poured into sat. Na₂CO₃-solution (30 mL). The aq. phase is extracted with CH₂Cl₂ (4×50 mL), the organic extracts are washed with 1M-NaH₂PO₄ (50 mL) and brine (50 mL), dried and evaporated. The residue is purified by flash chromatography (SiO₂, CH₂Cl₂-MeOH) to provide the title compound.

### C1.2. 1-(2-Methyl-quinolin-4-yl)-3-pyrrolidin-3-yl-urea dihydrochloride

A solution of 3-[3-(2-methyl-quinolin-4-yl)-ureido]-pyrrolidine-1-carboxylic acid tert-butyl ester (Example C1.1, 740 mg, 2 mmol) in dioxane (10 mL) is treated with 4M-HCl in dioxane (2 mL) for 3 h. The white precipitate is filtered, washed with ether and dried to provide the title compound as the dihydrochloride salt.

### C1.3. 1-(2-Methyl-quinolin-4-yl)-3-pyrrolidin-3-yl-urea

A solution of 1-(2-methyl-quinolin-4-yl)-3-pyrrolidin-3-yl-urea dihydrochloride (Example C1.2, 343.3 mg, 1 mmol) in MeOH (2 mL) is added to 1M-NaOH (10 mL) and the aq. phase extracted with CH₂Cl₂ (4×20 mL). The organic extracts are dried (Na₂SO₄), filtered and evaporated to provide the title compound.

Alternatively, the title compound can be prepared in racemic or enantiomerically pure form by hydrogenation of 1-(1-benzyl-pyrrolidin-3-yl)-3-(2-methyl-quinolin-4-yl)-urea (Examples 20.-22.) using the method described in Example 54.

### C2. 1-(2-Methyl-quinolin-4-yl)-3-piperidin-3-yl-urea

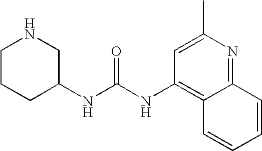

The title compound is prepared from 3-amino-piperidine-1-carboxylic acid tert-butyl ester (Example A4.) and 1,3-bis-(2-methyl-quinolin-4-yl)-urea (Example B2) using the method described in Example C1.

Preparation of Final Products

### Example 1

### 1-[1-(2,2-Diphenyl-ethyl)-pyrrolidin-3-yl-3-(2-methyl-quinolin-4-yl)-urea

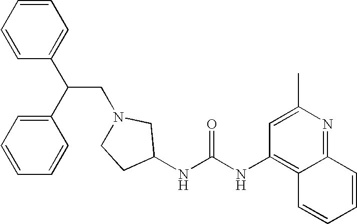

A solution of 1-(2-methyl-quinolin-4-yl)-3-pyrrolidin-3-yl-urea dihydrochloride (Example C1.2., 51.5 mg, 0.15 mmol), TEA (70 μL, 0.5 mmol), NaBHAC₃ (67 mg, 0.32 mmol) and diphenylacetaldehyde (36 μL, 0.20 mmol) in dry THF (1.5 mL) is stirred at r.t. for 15 h, then the solvent is evaporated and the residue purified by HPLC to provide the title compound.

### Example 2

### 1-[1-(1-Benzyl-2-phenyl-ethyl)-pyrrolidin-3-yl-3-(2-methyl-quinolin-4-yl)-urea

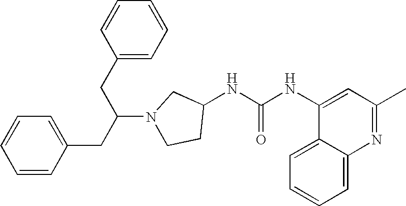

A solution of 1-(2-methyl-quinolin-4-yl)-3-pyrrolidin-3-yl-urea dihydrochloride (Example C1.2., 51.5 mg, 0.15 mmol), TEA (70 μL, 0.5 mmol), NaBHAc₃ (67 mg, 0.32 mmol) and dibenzylketone (42.1 mg, 0.2 mmol) in dry THF (1.5 mL) is stirred at r.t. for 15 h, then the solvent is evaporated and the residue purified by prep. HPLC to provide the title compound.

The following examples are prepared from the appropriate stereoisomer or the racemic mixture of Example C1.2 and commercially available aldehydes or, respectively, ketones using the method described in Example 1 or, respectively, Example 2.

| Example No | Example | t_{R} | [M + H]⁺ |
|---|---|---|---|
| 1. | 1-[1-(2,2-Diphenyl-ethyl)-pyrrolidin-3-yl]-3-(2-methyl- | 0.78 | 451.15 |
| | quinolin-4-yl)-urea | | |
| 2. | 1-[1-(1-Benzyl-2-phenyl-ethyl)-pyrrolidin-3-yl]-3-(2- | 0.79 | 465.26 |
| | methyl-quinolin-4-yl)-urea | | |
| 3. | 1-(2-Methyl-quinolin-4-yl)-3-(1-phenethyl-pyrrolidin- | 0.71 | 375.22 |
| | 3-yl)-urea | | |
| 4. | 1-(2-Methyl-quinolin-4-yl)-3-[1-(3-phenyl-propyl)- | 0.73 | 389.22 |
| | pyrrolidin-3-yl]-urea | | |
| 5. | 1-(2-Methyl-quinolin-4-yl)-3-(1-naphthalen-1- | 0.73 | 411.19 |
| | ylmethyl-pyrrolidin-3-yl)-urea | | |
| 6. | 1-(2-Methyl-quinolin-4-yl)-3-(1-naphthalen-2- | 0.73 | 411.21 |
| | ylmethyl-pyrrolidin-3-yl)-urea | | |
| 7. | 1-(1-Biphenyl-4-ylmethyl-pyrrolidin-3-yl)-3-(2-methyl- | 0.76 | 437.21 |
| | quinolin-4-yl)-urea | | |
| 8. | 1-(2-Methyl-quinolin-4-yl)-3-[1-(4-phenyl- | 0.71 | 429.39 |
| | cyclohexyl)-pyrrolidin-3-yl]-urea | | |
| 9. | 1-[(R)-1-(1-Methyl-2,2-diphenyl-ethyl)-pyrrolidin-3- | 0.71 | 465.42 |
| | yl]-3-(2-methyl-quinolin-4-yl)-urea | | |
| 10. | 1-[(S)-1-(1-Methyl-2,2-diphenyl-ethyl)-pyrrolidin-3- | 0.71 | 465.24 |
| | yl]-3-(2-methyl-quinolin-4-yl)-urea | | |

### Example 11

### 1-[1-(3,3-Diphenyl-propyl)-pyrrolidin-3-yl]-3-(2-methyl-quinolin-4-yl)-urea

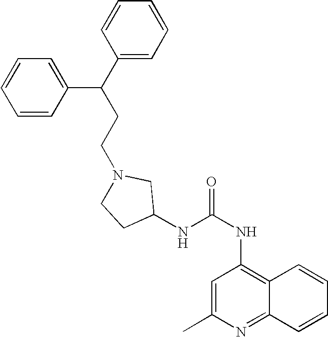

### Example 11.1

### 1-[1-(3,3-Diphenyl-propionyl)-pyrrolidin-3-yl]-3-(2-methyl-quinolin-4-yl)-urea

To a cooled (0° C.) mixture of 1-(2-methyl-quinolin-4-yl)-3-pyrrolidin-3-yl-urea dihydrochloride (Example C1.2., 172 mg, 0.5 mmol), 3,3-diphenylpropionic acid (135.8 mg, 0.6 mmol), HOBt (81 mg, 0.6 mmol), TEA (0.28 mL, 2 mmol) and a cat. amount of DMAP in CH₂Cl₂ (20 mL) is added EDC (115 mg, 0.6 mmol). The mixture is stirred at r.t. for 48 h. The mixture is quenched with sat. aq. Na₂CO₃ (25 mL), the phases are separated, and the aq. phase is extracted with CH₂Cl₂ (3×50 mL). The combined organic extracts are dried (Na₂SO₄), filtered and evaporated to provide the crude title compound.

### Example 11.2

### 1-[1-(3,3-Diphenyl-propyl)-pyrrolidin-3-yl]-3-(2-methyl-quinolin-4-yl)-urea

The crude 1-[1-(3,3-diphenyl-propionyl)-pyrrolidin-3-yl]-3-(2-methyl-quinolin-4-yl)-urea (Example 11.1.) is dissolved in THF (5 mL) and added to a cooled (0° C.) suspension of LiAlH₄ (100 mg, 2.5 mmol) in THF (20 mL). The mixture is warmed during 15 h to r.t. The reaction mixture is carefully added to EtOAc (100 mL) and MeOH (5 mL), and, subsequently, sat. aq. NaHCO₃ (2 mL) is added. The mixture is filtered, the filtercake washed with MeOH (2×50 mL), and the filtrate is evaporated. The residue is taken up in a minimal amount of MeOH, diluted with CH₂Cl₂, dried (Na₂SO₄), filtered and evaporated. The residue is purified by HPLC to provide the title compound.

The following examples are prepared from Example C1.2. or Example C2. and commercially available carboxylic acids using the method described in Example 11.

| Example | | | [M + |
|---|---|---|---|
| No | Example | t_{R} | H]⁺ |
| 11. | 1-[1-(3,3-Diphenyl-propyl)-pyrrolidin- | 0.73 | 465.16 |
| | 3-yl]-3-(2-methyl-quinolin-4-yl)-urea | | |
| 12. | 1-[1-(2,3-Diphenyl-propyl)-pyrrolidin-3- | 0.73 | 465.18 |
| | yl]-3-(2-methyl-quinolin-4-yl)-urea | | |
| 13. | 1-[1-(2-Hydroxy-2,2-diphenyl-ethyl)- | 0.69 | 467.16 |
| | pyrrolidin-3-yl]-3-(2-methyl-quinolin-4-yl)- | | |
| | urea | | |
| 14. | 1-[1-(2,2-Diphenyl-ethyl)-piperidin-3-yl]- | 0.71 | 465.43 |
| | 3-(2-methyl-quinolin-4-yl)-urea | | |
| 15. | 1-[1-(3,3-Diphenyl-propyl)-piperidin- | 0.74 | 479.26 |
| | 3-yl]-3-(2-methyl-quinolin-4-yl)-urea | | |

### Example 16

### 1-[(S)-1-(1-Benzyl-2-phenyl-ethyl)-pyrrolidin-3-yl]-3-(2-methyl-quinolin-4-yl)-urea

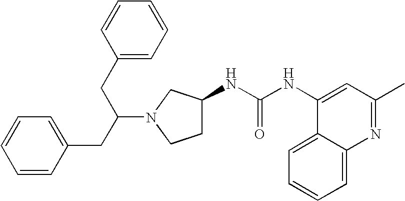

A suspension of (S)-1-(1-benzyl-2-phenyl-ethyl)-pyrrolidin-3-ylamine (Example A7., 70 mg, 0.25 mmol) and 1,3-bis-(2-methyl-quinolin-4-yl)-urea (Example B2, 86 mg 0.25 mmol) in MeOH (2 mL) is heated at reflux for 15 h. The solvent is evaporated and the residue purified by HPLC to provide the title compound.

The following examples are prepared from the appropriate stereoisomer or the racemic mixture of Example A1. or Examples A5.-A18. and Example B2. using the method described for Example 16.

| Example No | Example | t_{R} | [M + H]⁺ |
|---|---|---|---|
| 16. | 1-[(S)-1-(1-Benzyl-2-phenyl-ethyl)-pyrrolidin-3-yl]-3- | 0.73 | 465.27 |
| | (2-methyl-quinolin-4-yl)-urea | | |
| 17. | 1-[(R)-1-(1-Benzyl-2-phenyl-ethyl)-pyrrolidin-3-yl]-3- | 0.73 | 465.20 |
| | (2-methyl-quinolin-4-yl)-urea | | |
| 18. | 1-[(S)-1-(3,3-Diphenyl-propyl)-pyrrolidin-3-yl]-3-(2- | 0.73 | 465.22 |
| | methyl-quinolin-4-yl)-urea | | |
| 19. | 1-[(R)-1-(3,3-Diphenyl-propyl)-pyrrolidin-3-yl]-3-(2- | 0.73 | 465.23 |
| | methyl-quinolin-4-yl)-urea | | |
| 20. | (R)-1-(1-Benzyl-pyrrolidin-3-yl)-3-(2-methyl-quinolin- | 0.62 | 361.16 |
| | 4-yl)-urea | | |
| 21. | (S)-1-(1-Benzyl-pyrrolidin-3-yl)-3-(2-methyl-quinolin- | 0.62 | 361.14 |
| | 4-yl)-urea | | |
| 22. | 1-(1-Benzyl-pyrrolidin-3-yl)-3-(2-methyl-quinolin-4-yl)- | 0.69 | 361.14 |
| | urea | | |
| 23. | 1-[(S)-1-(2-Hydroxy-2,2-diphenyl-ethyl)-pyrrolidin-3- | 0.68 | 467.24 |
| | yl]-3-(2-methyl-quinolin-4-yl)-urea | | |
| 24. | 1-[(R)-1-(2-Hydroxy-2,2-diphenyl-ethyl)-pyrrolidin-3- | 0.68 | 467.24 |
| | yl]-3-(2-methyl-quinolin-4-yl)-urea | | |
| 25. | 1-[(S)-1-(1-Benzyl-2-phenyl-ethyl)-pyrrolidin-2- | 1.08 | 479.45 |
| | ylmethyl]-3-(2-methyl-quinolin-4-yl)-urea | | |
| 26. | 1-[(R)-1-(1-Benzyl-2-phenyl-ethyl)-pyrrolidin-2- | 1.08 | 479.45 |
| | ylmethyl]-3-(2-methyl-quinolin-4-yl)-urea | | |
| 27. | N,N-Diethyl-4-{(S)-3-[3-(2-methyl-quinolin-4-yl)- | 0.77 | 564.25 |
| | ureido]-pyrrolidin-1-yl}-2,2-diphenyl-butyramide | | |
| 28. | N,N-Diethyl-4-{(R)-3-[3-(2-methyl-quinolin-4-yl)- | 0.77 | 564.31 |
| | ureido]-pyrrolidin-1-yl}-2,2-diphenyl-butyramide | | |
| 29. | N,N-Dimethyl-4-{(S)-3-[3-(2-methyl-quinolin-4-yl)- | 0.73 | 536.24 |
| | ureido]-pyrrolidin-1-yl}-2,2-diphenyl-butyramide | | |
| 30. | N,N-Dimethyl-4-{(R)-3-[3-(2-methyl-quinolin-4-yl)- | 0.72 | 536.47 |
| | ureido]-pyrrolidin-1-yl}-2,2-diphenyl-butyramide | | |

### Example 31

### 1-(1-Biphenyl-3-ylmethyl-pyrrolidin-3-yl)-3-(2-methyl-quinolin-4-yl)-urea

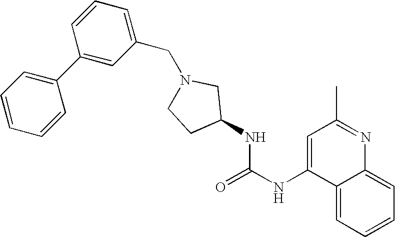

### Example 31.1

### 3-{3-[3-(2-Methyl-quinolin-4-yl)-ureido]-pyrrolidin-1-ylmethyl}-benzeneboronic acid

The title compound is prepared from 1-(2-methyl-quinolin-4-yl)-3-pyrrolidin-3-yl-urea (Example C1.) and 3-formyl-benzeneboronic acid using the method described in Example 1.

### Example 31.2

### 1-(1-Biphenyl-3-ylmethyl-pyrrolidin-3-yl)-3-(2-methyl-quinolin-4-yl)-urea

A mixture of 3-{3-[3-(2-methyl-quinolin-4-yl)-ureido]-pyrrolidin-1-ylmethyl}-benzeneboronic acid (139 mg, 0.34 mmol), 3M-aq. K₃PO₄ (1 mL), bromobenzene (63 mg, 0.4 mmol) and dioxane (2 mL) is saturated with argon and tetrakis-(triphenylphosphine)-palladium (20 mg, 1.7 mmol) is added. The mixture is heated at 100° C. for 15 h, cooled to r.t., quenched with sat. aq. Na₂CO₃ (10 mL) and extracted with CH₂Cl₂ (3×15 mL). The combined organic extracts are dried (Na₂SO₄), filtered and evaportated. The residue is purified by HPLC to provide the title compound.

| Example | | | [M + |
|---|---|---|---|
| No | Example | t_{R} | H]⁺ |
| 31. | 1-(1-Biphenyl-3-ylmethyl-pyrrolidin-3-yl)-3- | 0.70 | 437.29 |
| | (2-methyl-quinolin-4-yl)-urea | | |

### Example 32

### 1-((S)-1-Biphenyl-2-ylmethyl-pyrrolidin-3-yl)-3-(2-methyl-quinolin-4-yl)-urea

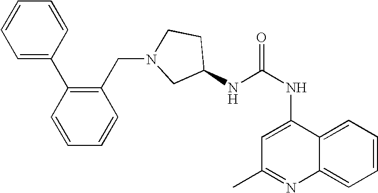

A mixture of 1-(2-methyl-quinolin-4-yl)-3-pyrrolidin-3-yl-urea dihydrochloride (Example C1.2., 172 mg, 0.5 mmol), 2-phenylbenzylbromide (148.3 mg, 0.6 mmol) and TEA (0.28 mL, 2 mmol) in THF (4 mL) is stirred at 65° C. for 15 h. The mixture is quenched with sat. aq. Na₂CO₃ (25 mL) and extracted with CH₂Cl₂ (3×50 mL). The combined organic extracts are dried (Na₂SO₄), filtered and evaporated. The residue is purified by HPLC to provide the crude title compound.

The following examples are pepared from the appropriate stereoisomer of Example C1. and commercially available bromides using the method described for Example 32.

| Example | | | |
|---|---|---|---|
| No | Example | t_{R} | [M + H]⁺ |
| 32. | 1-((S)-1-Biphenyl-2-ylmethyl-pyrrolidin- | 0.69 | 437.16 |
| | 3-yl)-3-(2-methyl-quinolin-4-yl)-urea | | |
| 33. | 1-[(S)-1-(3-Cyano-3,3-diphenyl-propyl)- | 0.74 | 490.23 |
| | pyrrolidin-3-yl]-3-(2-methyl-quinolin- | | |
| | 4-yl)-urea | | |
| 34. | 1-[(R)-1-(3-Cyano-3,3-diphenyl-propyl)- | 0.74 | 490.25 |
| | pyrrolidin-3-yl]-3-(2-methyl-quinolin- | | |
| | 4-yl)-urea | | |

### Example 35

### 1-(2,6-Dimethyl-pyridin-4-yl)-3-[(S)-1-(2,2-diphenyl-ethyl)-pyrrolidin-3-yl-urea

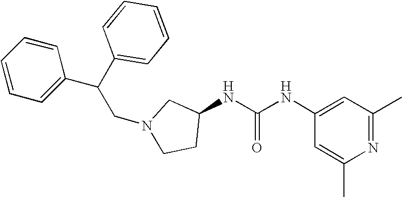

A suspension of (S)-1-(2,2-diphenyl-ethyl)-pyrrolidin-3-ylamine (Example A5., 66.6 mg, 0.25 mmol), TEA (35 μL, 0.25 mmol) and 1,3-bis-(2,6-dimethyl-pyridin-4-yl)-urea (Example B4., 67.5 mg 0.25 mmol) in dioxane (2 mL) is heated at reflux for 24 h. The solvent is evaporated and the residue purified by HPLC to provide the title compound.

The following examples are pepared from Examples A5.-A12. and Example B2. using the method described for Example 35.

| Exam- | | | |
|---|---|---|---|
| ple | | | [M + |
| No | Example | t_{R} | H]⁺ |
| 35. | 1-(2,6-Dimethyl-pyridin-4-yl)-3-[(S)-1-(2,2- | 0.68 | 415.41 |
| | diphenyl-ethyl)-pyrrolidin-3-yl]-urea | | |
| 36. | 1-[(S)-1-(1-Benzyl-2-phenyl-ethyl)-pyrrolidin- | 0.70 | 429.41 |
| | 3-yl]-3-(2,6-dimethyl-pyridin-4-yl)-urea | | |
| 37. | 1-[(R)-1-(1-Benzyl-2-phenyl-ethyl)-pyrrolidin- | 0.71 | 429.42 |
| | 3-yl]-3-(2,6-dimethyl-pyridin-4-yl)-urea | | |
| 38. | 1-(2,6-Dimethyl-pyridin-4-yl)-3-[(S)-1- | 0.66 | 431.18 |
| | (2-hydroxy-2,2-diphenyl-ethyl)-pyrrolidin-3- | | |
| | yl]-urea | | |
| 39. | 1-(2,6-Dimethyl-pyridin-4-yl)-3-[(R)-1- | 0.66 | 431.22 |
| | (2-hydroxy-2,2-diphenyl-ethyl)-pyrrolidin-3- | | |
| | yl]-urea | | |
| 40. | 1-(2,6-Dimethyl-pyridin-4-yl)-3-[(S)-1-(3,3- | 0.71 | 429.22 |
| | diphenyl-propyl)-pyrrolidin-3-yl]-urea | | |
| 41. | 1-(2,6-Dimethyl-pyridin-4-yl)-3-[(R)-1-(3,3- | 0.71 | 429.24 |
| | diphenyl-propyl)-pyrrolidin-3-yl]-urea | | |

### Example 42

### 1-[(S)-1-(3,3-Diphenyl-propyl)-pyrrolidin-3-yl-3-(2-ethyl-6-methyl-pyridin-4-yl)-urea

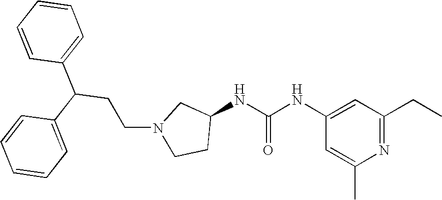

To a solution of (S)-1-(3,3-diphenyl-propyl)-pyrrolidin-3-ylamine (Example A9., 70 mg, 0.25 mmol) in CH₂Cl₂ is added a freshly prepared solution of 2-ethyl-4-isocyanato-6-methyl-pyridine (Example B8., 0.3 mmol) in toluene (2 mL). The mixture is stirred for 15 h at 20° C. Evaporation of the solvent and purification by HPLC provides the title compound.

The following examples are pepared from Examples A5.-A10. and Examples B5.-B10. using the method described for Example 42.

| Example No | Example | t_{R} | [M + H]⁺ |
|---|---|---|---|
| 42. | 1-[(S)-1-(3,3-Diphenyl-propyl)-pyrrolidin-3-yl]-3-(2- | 0.72 | 443.25 |
| | ethyl-6-methyl-pyridin-4-yl)-urea | | |
| 43. | 1-[(S)-1-(1-Benzyl-2-phenyl-ethyl)-pyrrolidin-3-yl]-3- | 0.72 | 443.24 |
| | (2-ethyl-6-methyl-pyridin-4-yl)-urea | | |
| 44. | 1-[(S)-1-(2,2-Diphenyl-ethyl)-pyrrolidin-3-yl]-3-(2- | 0.70 | 429.22 |
| | ethyl-6-methyl-pyridin-4-yl)-urea | | |
| 45. | 1-[(S)-1-(3,3-Diphenyl-propyl)-pyrrolidin-3-yl]-3-[2- | 0.80 | 517.45 |
| | methyl-6-((E)-styryl)-pyridin-4-yl]-urea | | |
| 46. | 1-[(S)-1-(2,2-Diphenyl-ethyl)-pyrrolidin-3-yl]-3-{2- | 0.79 | 521.42 |
| | [(E)-2-(4-fluoro-phenyl)-vinyl]-6-methyl-pyridin-4-yl}- | | |
| | urea | | |
| 47. | 1-[(S)-1-(2,2-Diphenyl-ethyl)-pyrrolidin-3-yl]-3-(2- | 0.77 | 505.41 |
| | methyl-6-phenethyl-pyridin-4-yl)-urea | | |
| 48. | 1-[(S)-1-(1-Benzyl-2-phenyl-ethyl)-pyrrolidin-3-yl]-3- | 0.74 | 457.43 |
| | (2-methyl-6-propyl-pyridin-4-yl)-urea | | |
| 49. | 1-[(S)-1-(2,2-Diphenyl-ethyl)-pyrrolidin-3-yl]-3-(2- | 0.71 | 443.39 |
| | methyl-6-propyl-pyridin-4-yl)-urea | | |
| 50. | 1-[(S)-1-(3,3-Diphenyl-propyl)-pyrrolidin-3-yl]-3-(2- | 0.73 | 457.42 |
| | methyl-6-propyl-pyridin-4-yl)-urea | | |
| 51. | 1-[2-(Benzyl-methyl-amino)-pyridin-4-yl]-3-[(S)-1- | 0.75 | 506.33 |
| | (2,2-diphenyl-ethyl)-pyrrolidin-3-yl]-urea | | |

### Example 52

### 1-[(S)-1-(3,3-Diphenyl-propyl)-pyrrolidin-3-yl]-3-(2-methyl-6-phenethyl-pyridin-4-yl)-urea

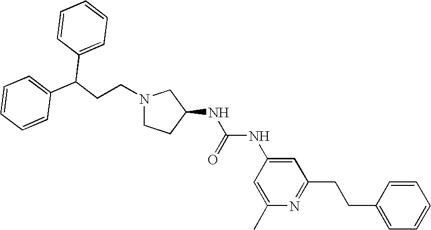

A suspension of 1-[(S)-1-(3,3-diphenyl-propyl)-pyrrolidin-3-yl]-3-[2-methyl-6-((E)-styryl)-pyridin-4-yl]-urea (Example 45., 10.4 mg, 0.02 mmol) and Pd—C 10% (10 mg) in MeOH (10 mL) is stirred under hydrogen atmosphere for 15 h. The catalyst is filtered off and the reaction mixture evaporated to provide the title compound.

The following compounds are prepared in an analogous fashion.

| Exam- | | | |
|---|---|---|---|
| ple | | | |
| No | Example | t_{R} | [M + H]⁺ |
| 52. | 1-[(S)-1-(3,3-Diphenyl-propyl)-pyrrolidin- | 0.79 | 519.50 |
| | 3-yl]-3-(2-methyl-6-phenethyl-pyridin-4-yl)- | | |
| | urea | | |
| 53. | 1-[(S)-1-(2,2-Diphenyl-ethyl)-pyrrolidin-3- | 0.78 | 523.48 |
| | yl]-3-{2-[2-(4-fluoro-phenyl)-ethyl]-6- | | |
| | methyl-pyridin-4-yl}-urea | | |

### Example 54

### 1-[(S)-1-(2,2-Diphenyl-ethyl)-pyrrolidin-3-yl-3-(2-methylamino-pyridin-4-yl)-urea

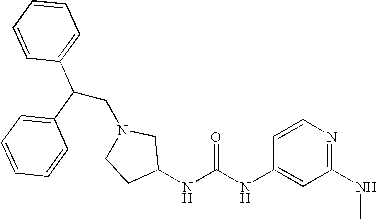

A suspension of 1-[2-(benzyl-methyl-amino)-pyridin-4-yl]-3-[(S)-1-(2,2-diphenyl-ethyl)-pyrrolidin-3-yl]-urea (Example 51., 151.7 mg, 0.3 mmol) and Pd—C 10% (50 mg) in MeOH (10 mL) is stirred at r.t. under hydrogen (7 bar) for 72 h. The catalyst is filtered off, the reaction mixture evaporated and the residue purified by HPLC to provide the title compound.

| Exam- | | | |
|---|---|---|---|
| ple | | | |
| No | Example | t_{R} | [M + H]⁺ |
| 54. | 1-[(S)-1-(2,2-Diphenyl-ethyl)-pyrrolidin-3- | 0.67 | 416.36 |
| | yl]-3-(2-methylamino-pyridin-4-yl)-urea | | |

### Example 55

### 1-[(S)-1-(2,2-Diphenyl-ethyl)-pyrrolidin-3-yl]-3-(2-propylamino-pyridin-4-yl)-urea

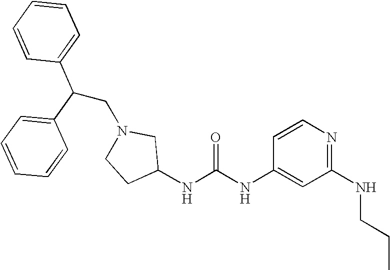

### Example 55.1

### 1-(2-Chloro-6-Propylamino-pyridin-4-yl)-3-[1-(2,2-diphenyl-ethyl)-pyrrolidin-3-yl-urea

The title compound is prepared from (S)-1-(2,2-diphenyl-ethyl)-pyrrolidin-3-ylamine (Example A5.) and (6-chloro-4-isocyanato-pyridin-2-yl)-propyl-amine (Example B11.) using the method described in Example 42.

### Example 55.2

### 1-[(S)-1-(2,2-Diphenyl-ethyl)-pyrrolidin-3-yl]-3-(2-propylamino-pyridin-4-yl)-urea

The title compound is prepared from 1-(2-chloro-6-propylamino-pyridin-4-yl)-3-[1-(2,2-diphenyl-ethyl)-pyrrolidin-3-yl]-urea using the method described in Example 52.

The following compounds are prepared in an analogous fashion.

| Exam- | | | |
|---|---|---|---|
| ple | | | |
| No | Example | t_{R} | [M+H]⁺ |
| 55. | 1-[(S)-1-(2,2-Diphenyl-ethyl)-pyrrolidin-3- | 0.71 | 444.34 |
| | yl]-3-(2-propylamino-pyridin-4-yl)-urea | | |
| 56. | 1-(2-Cyclopentylamino-pyridin-4-yl)-3-[(S)- | 0.74 | 470.22 |
| | 1-(2,2-diphenyl-ethyl)-pyrrolidin-3-yl]-urea | | |
| 57. | 1-(2-Benzylamino-pyridin-4-yl)-3-[(S)- | 0.74 | 492.35 |
| | 1-(2,2-diphenyl-ethyl)-pyrrolidin-3-yl]-urea | | |

### Example 58

### In Vitro Biological Characterization

The inhibitory activity of the compounds of general formula 1 on the actions of urotensin II can be demonstrated using the test procedures described hereinafter:

### 1) Inhibition of Human [¹²⁵I]-Urotensin II Binding to a Rhabdomyosarcoma Cell Line

Whole cell binding of human [¹²⁵I]-urotensin II is performed using human-derived TE-671 rhabdomyosarcoma cells (Deutsche Sammlung von Mikroorganismen und Zellkulturen, cell line #ACC-263), by methods adapted from a whole cell endothelin binding assay (Breu V et al., In vitro characterization of Ro-46-2005, a novel synthetic non-peptide antagonist of ET_{A} and ET_{B} receptors. FEBS Lett. 1993, 334, 210-214).

The assay is performed in 250 μL Dubecco's modified eagle medium, pH 7.4 (GIBCO BRL, CatNo 31885-023), including 25 mM HEPES (Fluka, CatNo 05473), 1.0% DMSO (Fluka, CatNo 41644) and 0.5% (w/v) BSA Fraction V (Fluka, CatNo 05473) in polypropylene microtiter plates (Nunc, CatNo 442587). 300'000 suspended cells are incubated with gentle shaking for 4 h at 20° C. with 20 pM human [¹²⁵I]Urotensin II (Anawa Trading SA, Wangen, Switzerland, 2130 Ci/mmol) and increasing concentrations of unlabeled antagonist. Minimum and maximum binding are derived from samples with and without 100 nM unlabelled U-II, respectively. After the 4 h incubation period, the cells are filtered onto GF/C filterplates (Packard, CatNo 6005174). The filter plates are dried, and then 50 μL scintillation cocktail (Packard, MicroScint 20, CatNo 6013621) is added to each well. The filterplates are counted in a microplate counter (Packard Bioscience, TopCount NXT).

All test compounds are dissolved and diluted in 100% DMSO. A ten-fold dilution into assay buffer is performed prior to addition to the assay. The final concentration of DMSO in the assay is 1.0%, which is found not to interfere with the binding. IC50 values are defined as the concentration of antagonist inhibiting 50% of the specific binding of [¹²⁵I]human U-II. Specific binding is the difference between maximum binding and minimum binding, as described above. An IC₅₀ value of 0.206 nM is found for unlabeled human U-II. The compounds of the invention are found to have IC₅₀ values ranging from 1 to 1000 nM in this assay.

### 2) Inhibition of Human Urotensin II-Induced Contractions on Isolated Rat Thoracic Aorta

Adult Wistar rats are anesthetized and exsanguinated. The thoracic aorta is excised, dissected and cut in 3-5 mm rings. The endothelium is removed by gentle rubbing of the intimal surface. Each ring is suspended in a 10 mL isolated organ bath filled with Krebs-Henseleit solution (in mM; NaCl 115, KCl 4.7, MgSO₄ 1.2, KH₂PO₄ 1.5, NaHCO₃ 25, CaCl₂ 2.5, glucose 10) kept at 37° C. and gassed with 95% O₂ and 5% CO₂. The rings are connected to force transducers and isometric tension is recorded (EMKA Technologies SA, Paris, France). The rings are stretched to a resting tension of 3 g. Cumulative doses of human urotensin II (10⁻¹² M to 10⁻⁶ M) are added after a 10 min incubation with the test compound or its vehicle. The functional inhibitory potency of the test compound is assessed by calculating the concentration ratio, i.e. the shift to the right of the EC₅₀ induced by a 10⁻⁵ M concentration of test compound. EC₅₀ is the concentration of urotensin needed to get a half-maximal contraction; pA₂ is the negative logarithm of the theoretical antagonist concentration which induces a two-fold shift in the EC₅₀ value.

1. Compounds A compound of the general formula 1, 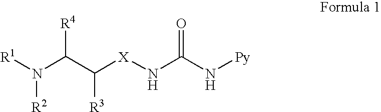
wherein:
Py represents quinolin-4-yl which is unsubstituted or mono- or disubstituted independently with lower alkyl or aryl-lower alkyl in the positions 2, 6 or 8; [1,8]naphthyridin-4-yl which is unsubstituted or monosubstituted in position 7 with lower alkyl; pyridin-4-yl which is unsubstituted or disubstituted in positions 2 and 6, wherein a substituent in position 2 is R⁵R⁶N—, lower alkyl, aryl-lower alkyl, or (E)-2-aryl-ethen-1-yl, and a substituent in position 6 is hydrogen or lower alkyl;
X is absent or represents a methylene group;
R¹ represents hydrogen; lower alkyl; aryl; aryl-lower alkyl; lower alkyl disubstituted with aryl; or lower alkyl disubstituted with aryl and additionally substituted at a carbon atom bearing an aryl group with OH, CN, or CONR⁷R⁸;
R² forms together with R³ a five-, six-, or seven-membered ring containing the nitrogen atom to which R² is attached as a ring atom and in which case R⁴ represents hydrogen; or
R² forms together with R⁴ a five-, six-, or seven-membered ring containing the nitrogen atom to which R² is attached as a ring atom and in which case R³ represents hydrogen;
the rings formed between R² and R³ or between R² and R⁴ are unsubstituted or monosubstituted with lower alkyl, aryl, aryl-lower alkyl, hydroxy, or aryloxy;
R⁵ and R⁶ independently represent hydrogen; lower alkyl; aryl; aryl-lower alkyl; or form together with the nitrogen atom to which they are attached a pyrrolidine, piperidine, or morpholine ring;
R⁷ and R⁸ independently represent hydrogen; lower alkyl; aryl; aryl-lower alkyl; or form together with the nitrogen atom to which they are attached a pyrrolidine, piperidine, or morpholine ring;
and optically pure enantiomers or diastereomers, mixtures of enantiomers or diastereomers, diastereomeric racemates, and mixtures of diastereomeric racemates; and their pharmaceutically acceptable salts, solvent complexes, and morphological forms.

2. The compound of claim 1, wherein R³ forms together with R² an unsubstituted five-, six-, or seven-membered ring containing the nitrogen atom to which R² is attached as a ring atom, and R⁴ is hydrogen.

3. The compound of claim 1, wherein R⁴ forms together with R² an unsubstituted five-, six-, or seven-membered ring containing the nitrogen atom to which R² is attached as a ring atom, and R³ is hydrogen.

4. The compound of claim 1, wherein Py represents quinolin-4-yl mono- or disubstituted independently with lower alkyl or aryl-lower alkyl in the positions 2 or 8.

5. The compound of claim 1, wherein Py represents pyridin-4-yl substituted in position 2 with R⁵R⁶N—, wherein R⁵ represents lower alkyl and R⁶ represents aryl-lower alkyl.

6. The compound of claim 1, wherein Py represents pyridin-4-yl substituted in position 2 with R⁵R⁶N—, wherein R⁶ represents hydrogen.

7. The compound of claim 1, wherein X is absent.

8. The compound of claim 1, wherein Py represents pyridin-4-yl disubstituted in position 2 and 6 with lower-alkyl.

9. The compound of claim 1 wherein Py represents pyridin-4-yl disubstituted in position 2 with aryl-lower alkyl and in position 6 with lower-alkyl.

10. The compound of claim 1, wherein R¹ represents lower alkyl disubstituted with aryl.

11. The compound of claim 1, wherein R¹ represents lower alkyl disubstituted with aryl and additionally substituted at a carbon atom bearing an aryl group with OH, CN, or CONR⁷R⁸.

12. The compound of claim 1, wherein X is absent, R³ forms together with R² an unsubstituted five-, six-, or seven-membered ring containing the nitrogen atom to which R² is attached as a ring atom, R⁴ is hydrogen, and Py represents quinolin-4-yl mono- or disubstituted independently with lower alkyl or aryl-lower alkyl in the positions 2 or 8.

13. The compound of claim 1, wherein X is absent, R³ forms together with R² an unsubstituted five-, six-, or seven-membered ring containing the nitrogen atom to which R² is attached as a ring atom, R⁴ is hydrogen, and Py represents pyridin-4-yl substituted in position 2 with R⁵R⁶N—, wherein R⁶ represents aryl-lower alkyl and R⁵ represents lower alkyl.

14. The compound of claim 1, wherein X is absent, R³ forms together with R² an unsubstituted five-, six-, or seven-membered ring containing the nitrogen atom to which R² is attached as a ring atom, R⁴ is hydrogen, and Py represents pyridin-4-yl substituted in position 2 with R⁵R⁶N—, wherein R⁶ represents hydrogen.

15. The compound of claim 1, wherein X is absent, R³ forms together with R² an unsubstituted five-, six-, or seven-membered ring containing the nitrogen atom to which R² is attached as a ring atom, R⁴ is hydrogen, and Py represents pyridin-4-yl disubstituted in position 2 and 6 with lower-alkyl.

16. The compound of claim 1, wherein X is absent, R³ forms together with R² an unsubstituted five-, six-, or seven-membered ring containing the nitrogen atom to which R² is attached as a ring atom, R⁴ is hydrogen, and Py represents pyridin-4-yl disubstituted in position 2 with aryl-lower alkyl and in position 6 with lower-alkyl.

17. The compound of claim 1, wherein X is absent, R³ forms together with R² an unsubstituted five-, six-, or seven-membered ring containing the nitrogen atom to which R² is attached as a ring atom, R⁴ is hydrogen, and R¹ represents lower alkyl disubstituted with aryl.

18. The compound of claim 1, wherein X is absent, R³ forms together with R² an unsubstituted five-membered ring containing the nitrogen atom to which R² is attached as a ring atom, R⁴ is hydrogen, and Py represents quinolin-4-yl monosubstituted with lower alkyl or aryl-lower alkyl in the position 2.

19. The compound of claim 1, wherein X is absent, R³ forms together with R² an unsubstituted five-membered ring containing the nitrogen atom to which R² is attached as a ring atom, R⁴ is hydrogen, and Py represents pyridin-4-yl substituted in position 2 with R⁵R⁶N—, wherein R⁶ represents hydrogen.

20. The compound of claim 1, wherein X is absent, R³ forms together with R² an unsubstituted five-membered ring containing the nitrogen atom to which R² is attached as a ring atom, R⁴ is hydrogen, and Py represents pyridin-4-yl disubstituted in position 2 and 6 with lower-alkyl.

21. The compound of claim 1, wherein X is absent, R³ forms together with R² an unsubstituted five-membered ring containing the nitrogen atom to which R² is attached as a ring atom, R⁴ is hydrogen, and R¹ represents lower alkyl disubstituted with aryl.

22. A compound selected from the group consisting of:
1-(2-Methyl-quinolin-4-yl)-3-pyrrolidin-3-yl-urea;
1-[1-(2,2-Diphenyl-ethyl)-pyrrolidin-3-yl]-3-(2-methyl-quinolin-4-yl)-urea;
1-[1-(1-Benzyl-2-phenyl-ethyl)-pyrrolidin-3-yl]-3-(2-methyl-quinolin-4-yl)-urea;
1-(2-Methyl-quinolin-4-yl)-3-(1-phenethyl-pyrrolidin-3-yl)-urea;
1-(2-Methyl-quinolin-4-yl)-3-[1-(3-phenyl-propyl)-pyrrolidin-3-yl]-urea;
1-(2-Methyl-quinolin-4-yl)-3-(1-naphthalen-1-ylmethyl-pyrrolidin-3-yl) -urea;
1-(2-Methyl-quinolin-4-yl)-3-(1-naphthalen-2-ylmethyl-pyrrolidin-3-yl)-urea;
1-(1-Biphenyl-4-ylmethyl-pyrrolidin-3-yl)-3-(2-methyl-quinolin-4-yl)-urea;
1-(2-Methyl-quinolin-4-yl)-3-[1-(4-phenyl-cyclohexyl)-pyrrolidin-3-yl]-urea;
1-[(R)-1-(1-Methyl-2,2-diphenyl-ethyl)-pyrrolidin-3-yl]-3-(2-methyl-quinolin-4-yl)-urea;
1-[(S)-1-(1-Methyl-2,2-diphenyl-ethyl)-pyrrolidin-3-yl]-3-(2-methyl-quinolin-4-yl)-urea;
1-[1-(3,3-Diphenyl-propyl)-pyrrolidin-3-yl]-3-(2-methyl-quinolin-4-yl)-urea;
1-[1-(2,3-Diphenyl-propyl)-pyrrolidin-3-yl]-3-(2-methyl-quinolin-4-yl)-urea;
1-[1-(2-Hydroxy-2,2-diphenyl-ethyl)-pyrrolidin-3-yl]-3-(2-methyl-quinolin-4-yl)-urea;
1-[1-(2,2-Diphenyl-ethyl)-piperidin-3-yl]-3-(2-methyl-quinolin-4-yl)-urea;
1-[1-(3,3-Diphenyl-propyl)-piperidin-3-yl]-3-(2-methyl-quinolin-4-yl)-urea;
1-[(S)-1-(1-Benzyl-2-phenyl-ethyl)-pyrrolidin-3-yl]-3-(2-methyl-quinolin-4-yl)-urea;
1-[(R)-1-(1-Benzyl-2-phenyl-ethyl)-pyrrolidin-3-yl]-3-(2-methyl-quinolin-4-yl)-urea;
1-[(S)-1-(3,3-Diphenyl-propyl)-pyrrolidin-3-yl]-3-(2-methyl-quinolin-4-yl)-urea;
1-[(R)-1-(3,3-Diphenyl-propyl)-pyrrolidin-3-yl]-3-(2-methyl-quinolin-4-yl)-urea;
(R)-1-(1-Benzyl-pyrrolidin-3-yl)-3-(2-methyl-quinolin-4-yl)-urea;
(S)-1-(1-Benzyl-pyrrolidin-3-yl)-3-(2-methyl-quinolin-4-yl)-urea;
1-(1-Benzyl-pyrrolidin-3-yl)-3-(2-methyl-quinolin-4-yl)-urea;
1-[(S)-1-(2-Hydroxy-2,2-diphenyl-ethyl)-pyrrolidin-3-yl]-3-(2-methyl-quinolin-4-yl)-urea;
1-[(R)-1-(2-Hydroxy-2,2-diphenyl-ethyl)-pyrrolidin-3-yl]-3-(2-methyl-quinolin-4-yl)-urea;
1-[(S)-1-(1-Benzyl-2-phenyl-ethyl)-pyrrolidin-2-ylmethyl]-3-(2-methyl-quinolin-4-yl)-urea;
1-[(R)-1-(1-Benzyl-2-phenyl-ethyl)-pyrrolidin-2-ylmethyl]-3-(2-methyl-quinolin-4-yl)-urea;
N,N-Diethyl-4-{(S)-3-[3-(2-methyl-quinolin-4-yl)-ureido]-pyrrolidin-1-yl}-2,2-diphenyl-butyramide;
N,N-Diethyl-4-{(R)-3-[3-(2-methyl-quinolin-4-yl)-ureido]-pyrrolidin-1-yl}-2,2-diphenyl-butyramide;
N,N-Dimethyl-4-{(S)-3-[3-(2-methyl-quinolin-4-yl)-ureido]-pyrrolidin-1-yl}-2,2-diphenyl-butyramide;
N,N-Dimethyl-4-{(R)-3-[3-(2-methyl-quinolin-4-yl)-ureido]-pyrrolidin-1-yl}-2,2-diphenyl-butyramide;
1-(1-Biphenyl-3-ylmethyl-pyrrolidin-3-yl)-3-(2-methyl-quinolin-4-yl)-urea;
1-((S)-1-Biphenyl-2-ylmethyl-pyrrolidin-3-yl)-3-(2-methyl-quinolin-4-yl)-urea;
1-[(S)-1-(3-Cyano-3,3-diphenyl-propyl)-pyrrolidin-3-yl]-3-(2-methyl-quinolin-4-yl)-urea;
1-[(R)-1-(3-Cyano-3,3-diphenyl-propyl)-pyrrolidin-3-yl]-3-(2-methyl-quinolin-4-yl)-urea;
1-[(S)-1-(1-Benzyl-2-phenyl-ethyl)-pyrrolidin-3-yl]-3-(2,6-dimethyl-pyridin-4-yl)-urea;
1-[(R)-1-(1-Benzyl-2-phenyl-ethyl)-pyrrolidin-3-yl]-3-(2,6-dimethyl-pyridin-4-yl)-urea;
1-(2,6-Dimethyl-pyridin-4-yl)-3-[(S)-1-(2,2-diphenyl-ethyl)-pyrrolidin-3-yl]-urea;
1-(2,6-Dimethyl-pyridin-4-yl)-3-[(S)-1-(2-hydroxy-2,2-diphenyl-ethyl)-pyrrolidin-3-yl]-urea;
1-(2,6-Dimethyl-pyridin-4-yl)-3-[(R)-1-(2-hydroxy-2,2-diphenyl-ethyl)-pyrrolidin-3-yl]-urea;
1-(2,6-Dimethyl-pyridin-4-yl)-3-[(S)-1-(3,3-diphenyl-propyl)-pyrrolidin-3-yl]-urea;
1-(2,6-Dimethyl-pyridin-4-yl)-3-[(R)-1-(3,3-diphenyl-propyl)-pyrrolidin-3-yl]-urea;
1-[(S)-1-(1-Benzyl-2-phenyl-ethyl)-pyrrolidin-3-yl]-3-(2-ethyl-6-methyl-pyridin-4-yl)-urea;
1-[(S)-1-(2,2-Diphenyl-ethyl)-pyrrolidin-3-yl]-3-(2-ethyl-6-methyl-pyridin-4-yl)-urea;
1-[(S)-1-(3,3-Diphenyl-propyl)-pyrrolidin-3-yl]-3-(2-ethyl-6-methyl-pyridin-4-yl)-urea;
1-[(S)-1-(3,3-Diphenyl-propyl)-pyrrolidin-3-yl]-3-[2-methyl-6-((E)-styryl)-pyridin-4-yl]-urea;
1-[(S)-1-(2,2-Diphenyl-ethyl)-pyrrolidin-3-yl]-3-{2-[(E)-2-(4-fluoro-phenyl)-vinyl]-6-methyl-pyridin-4-yl)-urea;
1-[(S)-1-(2,2-Diphenyl-ethyl)-pyrrolidin-3-yl]-3-(2-methyl-6-phenethyl-pyridin-4-yl)-urea;
1-[(S)-1-(1-Benzyl-2-phenyl-ethyl)-pyrrolidin-3-yl]-3-(2-methyl-6-propyl-pyridin-4-yl)-urea;
1-[(S)-1-(2,2-Diphenyl-ethyl)-pyrrolidin-3-yl]-3-(2-methyl-6-propyl-pyridin-4-yl)-urea;
1-[(S)-1-(3,3-Diphenyl-propyl)-pyrrolidin-3-yl]-3-(2-methyl-6-propyl-pyridin-4-yl)-urea;
1-[2-(Benzyl-methyl-amino)-pyridin-4-yl]-3-[(S)-1-(2,2-diphenyl-ethyl)-pyrrolidin-3-yl]-urea;
1-[(S)-1-(3,3-Diphenyl-propyl)-pyrrolidin-3-yl]-3-(2-methyl-6-phenethyl-pyridin-4-yl)-urea;
1-[(S)-1-(2,2-Diphenyl-ethyl)-pyrrolidin-3-yl]-3-{2-[2-(4-fluoro-phenyl)-ethyl]-6-methyl-pyridin-4-yl}-urea;
1-[(S)-1-(2,2-Diphenyl-ethyl)-pyrrolidin-3-yl]-3-(2-methylamino-pyridin-4-yl)-urea;
1-[(S)-1-(2,2-Diphenyl-ethyl)-pyrrolidin-3-yl]-3-(2-propylamino-pyridin-4-yl)-urea;
1-(2-Cyclopentylamino-pyridin-4-yl)-3-[(S)-1-(2,2-diphenyl-ethyl)-pyrrolidin-3-yl]-urea; and
1-(2-Benzylamino-pyridin-4-yl)-3-[(S)-1-(2,2-diphenyl-ethyl)-pyrrolidin-3-yl]-urea.

23. A pharmaceutical composition comprising a compound of claim 1 and a pharmaceutically acceptable carrier or adjuvant, or both.

24. (canceled)

25. (canceled)

26. (canceled)

27. (canceled)

28. The pharmaceutical composition of claim 23 further comprising an additional pharmacologically active compound.

29. The pharmaceutical composition of claim 28, wherein the additional pharmacologically active compound is selected from the group consisting of ACE inhibitors, angiotensin II receptor antagonists, endothelin receptor antagonists, vasopressin antagonists, beta-adrenergic antagonists, alpha-adrenergic antagonists, vasopressin antagonists, TNFalpha antagonists, and peroxisome proliferator activator receptor modulators.

30. A method of preventing or treating a disorder which is associated with a dysregulation of urotensin II or urotensin II receptors, comprising administering to a subject in need thereof a therapeutically effective amount of the compound of claim 1.

31. The method of claim 30, wherein the disorder is at selected from the group consisting of hypertension, atherosclerosis, angina, myocardial ischemia, congestive heart failure, cardiac insufficiency, cardiac arrhythmias, renal ischemia, chronic kidney disease, renal failure, stroke, cerebral vasospasm, cerebral ischemia, dementia, migraine, subarachnoidal hemorrhage, diabetes, diabetic arteriopathy, diabetic nephropathy, connective tissue diseases, cirrhosis, asthma, chronic obstructive pulmonary disease, high-altitude pulmonary edema, Raynaud's syndrome, portal hypertension, thyroid dysfunction, pulmonary edema, pulmonary hypertension, and pulmonary fibrosis.

32. A method of preventing or treating a disorder comprising administering to a subject in need thereof a prophylactically or therapeutically effective amount of the compound of claim 1, wherein the disorder is selected from the group consisting of restenosis after balloon or stent angioplasty, cancer, prostatic hypertrophy, erectile dysfunction, hearing loss, amaurosis, chronic bronchitis, asthma, gram negative septicemia, shock, sickle cell anemia, glomerulonephritis, renal colic, glaucoma, therapy and prophylaxis of diabetic complications, complications of vascular or cardiac surgery or after organ transplantation, complications of cyclosporin treatment, pain, addiction, schizophrenia, Alzheimer's disease, anxiety, obsessive-compulsive behavior, seizures, stress, and depression.

33. A method of preventing or treating a disorder, comprising administering to a subject in need thereof a therapeutically effective amount of the pharmaceutical composition of claim 23, wherein the disorder is selected from the group consisting of hypertension, atherosclerosis, angina or myocardial ischemia, congestive heart failure, cardiac insufficiency, cardiac arrhythmias, renal ischemia, chronic kidney disease, renal failure, stroke, cerebral vasospasm, cerebral ischemia, dementia, migraine, subarachnoidal hemorrhage, diabetes, diabetic arteriopathy, diabetic nephropathy, connective tissue diseases, cirrhosis, asthma, chronic obstructive pulmonary disease, high-altitude pulmonary edema, Raynaud's syndrome, portal hypertension, thyroid dysfunction, pulmonary edema, pulmonary hypertension, or pulmonary fibrosis, restenosis after balloon or stent angioplasty, cancer, prostatic hypertrophy, erectile dysfunction, hearing loss, amaurosis, chronic bronchitis, asthma, gram negative septicemia, shock, sickle cell anemia, glomerulonephritis, renal colic, glaucoma, therapy and prophylaxis of diabetic complications, complications of vascular or cardiac surgery or after organ transplantation, complications of cyclosporin treatment, pain, addiction, schizophrenia, Alzheimer's disease, anxiety, obsessive-compulsive behavior, seizures, stress, and depression.

34. The method of claim 33, wherein the pharmaceutical composition further comprises an additional pharmacologically active compound selected from the group consisting of ACE inhibitors, angiotensin II receptor antagonists, endothelin receptor antagonists, vasopressin antagonists, beta-adrenergic antagonists, alpha-adrenergic antagonists, vasopressin antagonists, TNFalpha antagonists, and peroxisome proliferator activator receptor modulators.
